(19) United States
(12) Patent Application Publication
(10) Pub. No.: US 2022/0273748 A1
(43) Pub. Date: Sep. 1, 2022

(54) PLANT FERMENT AND WEIGHT LOSS METHOD FOR USING THE SAME

(71) Applicant: TCI CO., LTD., Taipei (TW)

(72) Inventors: YUNG-HSIANG LIN, Taipei (TW); PEI-YI WU, Taipei (TW); HUAN-YOU LIN, Taipei (TW)

(21) Appl. No.: 17/680,330

(22) Filed: Feb. 25, 2022

Related U.S. Application Data
(60) Provisional application No. 63/153,955, filed on Feb. 26, 2021.

(51) Int. Cl. A61K 36/605; A61K 36/60; A61K 36/185; A61K 36/42; A61K 36/235

(52) U.S. Cl. CPC A61K 36/605; A61K 36/60; A61K 36/185; A61K 36/42; A61K 36/235

(57) ABSTRACT

Provided is a plant ferment, prepared by: mixing a mulberry (Morus alba), a pomegranate (Punica granatum), a purslane (Portulaca oleracea), a wild bitter melon (Momordica charantia var. abbreviata), and a fennel (Foeniculum vulgare) in a ratio of (0.5-4):(4-8):(0.5-4):(0.1-2):(0.5-4) to form a mixture; extracting the mixture with a solvent to obtain a plant extract; and fermenting the plant extract to obtain the plant ferment.

### CROSS-REFERENCE TO RELATED APPLICATION

This application claims the benefit of U.S. provisional application Ser. No. 63/153,955, filed on Feb. 26, 2021. The entirety of the above-mentioned patent application is hereby incorporated by reference herein and made a part of the specification.

### REFERENCE OF AN ELECTRONIC SEQUENCE LISTING

The contents of the electronic sequence listing (P211770USI_ST25.txt; Size: 3.11 KB; and Date of Creation: Feb. 24, 2022) is herein incorporated by reference in its entirety.

### BACKGROUND

### Technical Field

The present invention relates to a plant ferment and a weight loss method with the same, and in particular, to a plant ferment prepared from a mulberry (Morus alba), a pomegranate (Punica granatum), a purslane (Portulaca oleracea), a wild bitter melon (Momordica charantia var. abbreviata), and a fennel (Foeniculum vulgare), having the use of weight loss.

### Related Art

With the changes of the times, people pursue the perfect body, and have high requirements for the appearance, skin texture, body shape, and posture from the outside, and the health, body fat, and metabolic rate from the inside. A healthy body shape and posture is a major factor in maintaining a beautiful appearance. Therefore, people pay more and more attention to the health care and nurture of the body, to maintain the best state from the inside to the outside.

To resolve the foregoing problem, there is an urgent need for a person skilled in the art to develop functional foods to resolve the foregoing problem, so as to benefit the vast population in need thereof.

### SUMMARY

In view of this, provided is a plant ferment prepared from a mulberry (Morus alba), a pomegranate (Punica granatum), a purslane (Portulaca oleracea), a wild bitter melon (Momordica charantia van: abbreviata), and a fennel (Foeniculum vulgare), having the function of weight loss.

In some embodiments, a plant ferment is prepared by: mixing a mulberry (Morus alba), a pomegranate (Punica granatum), a purslane (Portulaca oleracea), a wild bitter melon (Momordica charantia var. abbreviata), and a fennel (Foeniculum vulgare) in a ratio of (0.5-4):(4-8):(0.5-4):(0.1-2):(0.5-4) to form a mixture; extracting the mixture with a solvent to obtain a plant extract; and fermenting the plant extract to obtain the plant fermented product.

In some embodiments, the plant extract is obtained by extracting the mixture including the mulberry, the pomegranate, the purslane, the wild bitter melon, and the fennel with water as the solvent at 50-100° C. for 0.5-2 h.

In some embodiments, the mixture and water are mixed in a ratio of (10-15):(80-90).

In some embodiments, the plant ferment is obtained by fermenting the plant extract with a yeast (Saccharomyces cerevisiae), a Lactobacillus plantarum, and an Acetobacter aceti.

In some embodiments, the yeast is added in an amount of 0.01-0.5% (w/w); the Lactobacillus plantarum is added in an amount of 0.01-0.2% (w/w); and the Acetobacter aceti is added in an amount of 1-10% (w/w).

In some embodiments, a plant ferment is used for preparing a composition for weight loss. The plant ferment is prepared by: mixing a mulberry (Morus alba), a pomegranate (Punica granatum), a purslane (Portulaca oleracea), a wild bitter melon (Momordica charantia var. abbreviata), and a fennel (Foeniculum vulgare) in a ratio of (0.5-4):(4-8):(0.5-4):(0.1-2):(0.5-4) to form a mixture; extracting the mixture with a solvent to obtain a plant extract; and fermenting the plant extract to obtain the plant ferment.

In some embodiments, a weight loss method includes administering to a subject in need thereof an effective dose of composition, where the composition includes a plant ferment. The plant ferment is prepared by: mixing a mulberry (Morus alba), a pomegranate (Punica granatum), a purslane (Portulaca oleracea), a wild bitter melon (Momordica charantia var. abbreviata), and a fennel (Foeniculum vulgare) in a ratio of (0.5-4):(4-8):(0.5-4):(0.1-2):(0.5-4) to form a mixture; extracting the mixture with a solvent to obtain a plant extract; and fermenting the plant extract to obtain the plant ferment.

In some embodiments, the composition is capable of reducing the appetite, increasing the time between meals, reducing hunger, and/or reducing the number of meals other than dinner per week.

In some embodiments, the composition is capable of reducing body weight, body mass index (BMI), whole body fat, and/or hip circumference.

In some embodiments, the composition is capable of reducing insulin resistance.

In some embodiments, the plant ferment increases an expression level of fat metabolism genes in cells and/or reduces an expression level of fat accumulation genes.

In some embodiments, the fat metabolism gene is selected from ATGL, LIPE, UCP1, UCP2, and a combination thereof.

In some embodiments, the fat accumulation gene is PLIN1 and/or PPARG2.

In some embodiments, the plant ferment is further prepared into a food composition, a dietary supplement composition, or a skin topical agent.

Based on the above, the plant ferment of any embodiment is obtained by fermenting the plant extract containing biologically active ingredients extracted from a mulberry, a pomegranate, a purslane, a wild bitter melon, and a fennel with a yeast, a Lactobacillus plantarum, and an Acetobacter aceti, and can be used to prepare a composition for weight loss. In some embodiments, the composition is effectively involved in regulating the process of eating and fat storage of a subject, and can reduce the appetite of the subject, increase the time between meals of the subject, reduce hunger of the subject, and/or reduce the number of meals other than dinner of the subject per week. In some embodiments, the composition contributes to the effective reduction of fat stored in cells by increasing an expression level of fat metabolism genes in cells and/or reducing an expression level of fat accumulation genes, thereby achieving the effects of reducing body weight, BMI, whole body fat, and/or hip circumference. In some embodiments, the composition may also reduce insulin resistance, and increase the sensitivity of body cells to glucose, so that excess energy is less likely to be stored as fat, to reduce fat accumulation. Based on this, the plant ferment of any embodiment can lose fat, reduce the appetite, increase the time between meals, reduce hunger, reduce the number of meals other than dinner per week, reduce insulin resistance, and reduce fat stored in cells, thereby achieving the effects of reducing body weight, BMI, whole body fat, and/or hip circumference.

### BRIEF DESCRIPTION OF THE DRAWINGS

FIG. 1 is a graph showing experimental results of the expression level of fat metabolism genes, where “*” represents a p value less than 0.05, “**” represents a p value less than 0.01, and “***” represents a p value less than 0.001;

FIG. 2 is a graph showing experimental results of the expression level of fat accumulation genes, where “***” represents a p value less than 0.001;

FIG. 3 is a graph showing experimental results of lipid droplet accumulation, where “*” represents a p value less than 0.05;

FIG. 4 is a graph showing experimental results of “difficulty in controlling appetite”, “short time between meals”, “frequent feeling of hunger”, and “high overall appetite level” of subjects at week 0, week 2, and week 4, where compared with week 0, “*” represents a p value less than 0.05, and “**” represents a p value less than 0.01;

FIG. 5 is a graph showing experimental results of the number of subjects who feel improvement in FIG. 4;

FIG. 6 is a graph showing experimental results of “the average number of meals other than dinner per week” of subjects at week 0, week 2, and week 4;

FIG. 7 is a graph showing experimental results of “satisfaction on the appetite suppressant effect” of subjects at week 2 and week 4;

FIG. 8 is a graph showing data analysis of the average weight of subjects drinking the plant ferment at week 0 and week 4;

FIG. 9 is a graph showing data analysis of the average BMI of subjects drinking the plant ferment at week 0 and week 4;

FIG. 10 is a graph showing data analysis of the average whole body fat percentage of subjects drinking the plant ferment at week 0 and week 4;

FIG. 11 is a graph showing data analysis of the average hip circumference of subjects drinking the plant ferment at week 0 and week 4;

FIG. 12 is a graph showing data analysis of the average insulin resistance index of subjects drinking the plant ferment at week 0 and week 4;

FIG. 13 is HPLC fingerprints of the plant aqueous extract and the plant ferment;

FIG. 14 is the ¹H-NMR spectrum of the compound TCI-LFT-01;

FIG. 15 is the ¹H-NMR spectrum of the compound TCI-LFT-02;

FIG. 16 is the ¹H-NMR spectrum of the compound TCI-LFT-03;

FIG. 17 is the ¹H-NMR spectrum of the compound TCI-LFT-04;

FIG. 18 is the ¹H-NMR spectrum of the compound TCI-LFT-05;

FIG. 19 is the ¹H-NMR spectrum of the compound TCI-LFT-06;

FIG. 20 is the ¹H-NMR spectrum of the compound TCI-LFT-07;

FIG. 21 is the ¹H-NMR spectrum of the compound TCI-LFT-08;

FIG. 22 is the ¹H-NMR spectrum of the compound TCI-LFT-09;

FIG. 23 is the ¹H-NMR spectrum of the compound TCI-LFT-10;

FIG. 24 is the ¹H-NMR spectrum of the compound TCI-LFT-11;

FIG. 25 is the ¹H-NMR spectrum of the compound TCI-LFT-12;

FIG. 26 is the ¹H-NMR spectrum of the compound TCI-LFT-13;

FIG. 27 is the ¹H-NMR spectrum of the compound TCI-LFT-14;

FIG. 28 is the ¹H-NMR spectrum of the compound TCI-LFT-15;

FIG. 29 is the ¹³C-NMR spectrum of the compound TCI-LFT-15;

FIG. 30 is a graph showing experimental results of 9 compounds on the relative gene expression level of the PYY gene; and

FIG. 31 is a graph showing experimental results of 5 compounds on lipid droplet accumulation.

### DETAILED DESCRIPTION

The following will describe some specific implementations of the instant disclosure. Without departing from the spirit of the instant disclosure, the instant disclosure can still be practiced in many different forms, and the protection scope should not be limited to the conditions specified in this specification.

Numerical values used herein are approximate values, and all experimental data are expressed within the range of ±10%, and best within the range of ±5%. Statistical analysis is conducted by using Excel software. Data is expressed as mean t standard deviation (SD), and the differences between groups are analyzed by student's t-test.

The term “extract” refers to a product prepared through extraction. The extract may be a solution obtained by dissolving a solute in a solvent, or may be a solvent-free or substantially solvent-free concentrate or essence.

In the description of the following embodiments, unless otherwise specified, the symbol “%” refers to weight percentage.

In some embodiments, five raw materials, including a mulberry, a pomegranate, a purslane, a wild bitter melon, and a fennel, are mixed in a specific ratio to form a mixture, the mixture is extracted with a solvent at a specific temperature for a proper time to obtain a plant extract, and the plant extract is fermented to obtain a plant ferment.

Mulberry (Morus alba) is the fruit of the mulberry tree, a perennial woody plant of the genus Morus of the family Moraceae, which is oval, 1-3 cm long, and has an uneven surface. In addition, the fruit is green when immature, gradually grows into white and red, and becomes purple-red or purple-black when mature, with a sweet and sour taste. In some embodiments, active ingredients (such as rutin, anthocyanidin, and resveratrol) of mulberry may be extracted from the mixture with the solvent, and a subject after taking the plant ferment containing the active ingredients of mulberry can achieve various effects such as anti-cancer, anti-aging, anti-inflammatory, increasing blood vessel elasticity, protecting eyes, helping digestion, and preventing gray hair.

Pomegranate (Punica granatum) is the fruit of the genus Punica of the family Lythraceae. The pomegranate fruit is nearly spherical, with a thick peel, a persistent calyx at the top, and is about 6 cm in diameter. In addition, the edible part of the pomegranate fruit is a fleshy outer seed coat, bright red, light red, or white, juicy, sweet and sour, and has the effect of astringency, anti-diarrhea, hemostasis, and insecticide.

Purslane (Portulaca oleracea, also known as pursley) is a plant of the genus Portulaca of the family Portulacaceae, and is an annual herb. The whole plant of purslane is thick and succulent, usually prostrate and glabrous; the cylindrical stem is prostrate at the lower part and is slightly erect at the upper part; and the stem is divergent, with a purple-red color. Thick obovate-cuneate leaves are opposite, with rounded apex, entire; small yellowish flowers in summer; conical capsules, cover cracked. In addition, purslane has the effects of clearing away heat and removing toxic substances, dispersing blood stasis and reducing swelling, dehumidification and relieving dysentery, diuresis and moisturizing the lung, and quenching thirst and helping produce saliva.

Wild bitter melon (Momordica charantia var. abbreviata) is an annual climbing herb of the genus Momordica of the family Cucurbitaceae. Wild bitter melon has lush branches, its vines have tendrils and are hairy and climbable, and the whole plant has a specific odor. The shape of wild bitter melon is smaller than that of common cultivars. The color of the fruit ranges from green to dark green. The fruit is 3-15 cm in length and 2-4 cm in width. The shape of the fruit ranges from olive to oblong. The fruit surface has rib-like protrusions, some of which are spiny. Wild bitter melon contains charantin, leading to a bitter taste, which turns bitter and sweet after cooking. It has the effects of promoting appetite, quenching thirst, cooling, detoxifying, and expelling cold.

Fennel (Foeniculum vulgare) is a flowering plant species in the genus Foeniculum of the family Apiaceae. Fennel is a cold-resistant perennial herb with a length of up to 2.5 m high. It is pink-green straight, with hollow stems and leaves up to 40 cm long. The tail of fennel is elongated, about 0.5 mm wide. The flower pattern of fennel is in a form of 5-15 cm wide umbels with 20-50 tiny yellow flowers per umbel. The fruit of fennel is 4-10 mm long, 0.5 mm wide or less, having pink-green seeds in shallow grooves on the surface.

Herein, the raw materials used including a mulberry, a pomegranate, a purslane, a wild bitter melon, and a fennel usually refer to the fruit, leaves, flowers, stems, roots, or whole plant, which may be raw, dried, or processed by other physical methods to facilitate handling, and may further be whole, chopped, diced, milled, ground, or processed by other methods to affect the size and physical integrity of the raw material.

For example, the raw material of mulberry used may be a mulberry fruit or a mulberry juice squeezed from the fruit, the raw material of pomegranate used may be a pomegranate juice squeezed from a pomegranate pulp or an edible part (without the peel) of a pomegranate fruit, the raw material of purslane used may be leaves of the purslane, the raw material of wild bitter melon used may be a fruit of the wild bitter melon, and the raw material of fennel used may be a fruit of the fennel or a powder prepared from the fruit of the fennel.

In some embodiments, the plant extract may be a plant juice obtained by squeezing the raw materials including a mulberry, a pomegranate, a purslane, a wild bitter melon, and a fennel. In some other embodiments, the plant extract may be obtained by soaking the raw materials including a mulberry, a pomegranate, a purslane, a wild bitter melon, and a fennel in water to be extracted at room temperature for a proper time, and filtering to remove solid impurities. In still some other embodiments, the plant extract may be obtained by crushing and squeezing the raw materials including a mulberry, a pomegranate, a purslane, a wild bitter melon, and a fennel, being extracted with a solvent, removing pomace and fine suspended substances, and then concentrating it to a specific sugar content. For example, the sugar content is 9-11° Bx. Adequate sugar can ensure the smooth progress of subsequent fermentation to ensure that the strains have adequate nutrients for subsequent fermentation.

In some embodiments, the five raw materials of mulberry, pomegranate, purslane, wild bitter melon, and fennel are in a weight ratio of (0.5-4):(4-8):(0.5-4):(0.1-2):(0.5-4). For example, the five raw materials of mulberry, pomegranate, purslane, wild bitter melon, and fennel are in a weight ratio of 6:2:2:2:1.

In some embodiments, the solvent may be water or alcohol, and the mixture and the solvent are in a weight ratio of (10-15):(80-90). For example, the mulberry, the pomegranate, the purslane, the wild bitter melon, the fennel, and the solvent are in a weight ratio of 6:2:2:2:1:90.

In some embodiments, the mixture also includes glucose. For example, 1-5% glucose is added into a mixed liquid of mulberry, pomegranate, purslane, wild bitter melon, and fennel in a weight ratio of (0.5-4):(4-8):(0.5-4):(0.1-2):(0.5-4) to form a mixture, and the mixture is extracted at a specific temperature for a proper time to obtain a plant extract. Herein, the glucose and the mixed liquid of mulberry, pomegranate, purslane, wild bitter melon, and fennel are subjected to extraction together, so as to help the glucose dissolve and avoid contamination.

In some embodiments, the extraction means that the mixture is extracted at room temperature for a specific time, or the mixture is maintained at 50-100° C., standing for 0.5-1.5 h. For example, the extraction means that the mixture is maintained at 95° C., standing for 1 h.

In some embodiments, the fermentation means that the fermenting procedure is carried out with a plurality of strains in order. Specifically, the plant extract is used as a culture medium for subsequent fermentation, and the culture medium is fermented with a plurality of strains for 4-15.5 days to obtain a fermented stock solution. Relative to the culture medium, the plurality of strains include 0.01-0.5% of yeast, 0.01-0.2% of lactic acid bacteria, and 1-10% of acetic acid bacteria.

In some embodiments, the strains are directly added into the culture medium for fermentation without filtering out solid substances (that is, the mulberry, pomegranate, purslane, wild bitter melon, and fennel remaining after extraction) therein, so as to further extract active ingredients from the solid substances by using the strains.

The yeast may be commercially available Saccharomyces cerevisiae. For example, the yeast may be Saccharomyces cerevisiae with a deposit number BCRC20271 (an international deposit number ATCC26602) purchased from the Food Industry Research and Development Institute.

The lactic acid bacteria may be commercially available Lactobacillus plantarum or commercially available Streptococcus thermophilus. For example, the lactic acid bacteria may be Lactobacillus plantarum (with a deposit number BCRC910760 purchased from the Food Industry Research and Development Institute) or Streptococcus thermophilus TC1378 with a deposit number BCRC910760 (an international deposit number DSM32451).

The acetic acid bacteria may be commercially available Acetobacter aceti. For example, the lactic acid bacteria may be Acetobacter aceti with a deposit number BCRC11688 (an international deposit number ATCC15973) purchased from the American Type Culture Collection.

In some embodiments, the “Saccharomyces cerevisiae”, “Lactobacillus plantarum”, and “Acetobacter aceti” are intended to cover, respectively, Saccharomyces cerevisiae, Lactobacillus plantarum, and Acetobacter aceti that are readily available to those skilled in the art (for example, available from domestic or foreign depositories), or may be Saccharomyces cerevisiae, Lactobacillus plantarum, and Acetobacter aceti that are obtained through isolation and purification from natural sources by the commonly used microbial isolation methods in the art.

In some embodiments, 0.01-0.5% of yeast is added into the plant extract to ferment for 12-36 h to form a first initial fermentation broth. Based on this, the yeast is first added into the plant extract to produce alcohol during the fermentation, which helps extract different active ingredients from the mulberry, pomegranate, purslane, wild bitter melon, and fennel. In addition, during the fermentation, the pH value of the plant extract gradually decreases, so that a solution environment with different pH values may be provided to help extract different active ingredients from the mulberry, pomegranate, purslane, wild bitter melon, and fennel. In some embodiments, the first initial fermentation broth has a pH value less than 4 and a sugar content about 10° Bx.

Then, 0.01-0.2% of lactic acid bacteria is added into the first initial fermentation broth to ferment for 12-36 h to form a second initial fermentation broth. Based on this, the lactic acid bacteria are added into the first initial fermentation broth to further consume glucose therein to reduce the sugar content and to produce lactic acid to reduce the pH value of the first initial fermentation broth. In addition, the decrease in pH value helps further extract different active ingredients from the mulberry, pomegranate, purslane, wild bitter melon, and fennel.

1-10% of acetic acid bacteria are added into the second initial fermentation broth to ferment for 3-10 days to form a third initial fermentation broth. Based on this, the acetic acid bacteria are added into the second initial fermentation broth to consume alcohol therein and further reduce the content of glucose.

In some embodiments, the third initial fermentation broth is filtered and concentrated to obtain a plant fermented stock solution. For example, the third initial fermentation broth may be filtered by a 200-mesh filter, and may be concentrated under reduced pressure at 60±5° C.

In some embodiments, after the third initial fermentation broth is filtered and concentrated under reduced pressure to obtain the plant fermented stock solution, water is added into it to supplement the weight reduced through the concentration under reduced pressure to obtain a plant ferment.

In some embodiments, oligosaccharides are added into the plant fermented stock solution to make the sugar content to 28° Bx to form a plant ferment. For example, the oligosaccharide refers to a saccharide polymer containing 3-10 monosaccharides. The oligosaccharides may be fructooligosaccharides, galacto-oligosaccharides, xylo-oligosaccharides, isomalto-oligosaccharides, and the like. In some embodiments, the oligosaccharides added may be an oligosaccharide solution containing 40-70% of isomalto-oligosaccharides.

It can be learned that a method for preparing a plant ferment includes: mixing raw materials of mulberry, pomegranate, purslane, wild bitter melon, and fennel in a weight ratio of (0.5-4):(4-8).(0.5-4):(0.1-2):(0.5-4) to form a mixture, extracting the mixture with a solvent at 50-100° C. for 0.5-1.5 h to obtain a plant extract, fermenting the plant extract with 0.01-0.5% of Saccharomyces cerevisiae for 12-36 h to form a first initial fermentation broth, adding 0.01-0.2% of Lactobacillus plantarum into the first initial fermentation broth to ferment for 12-36 h to form a second initial fermentation broth, adding 1-10% of Acetobacter aceti into the second initial fermentation broth to ferment for 3-10 days to form a third initial fermentation broth, and concentrating the third initial fermentation broth under reduced pressure to formulate the plant ferment.

In some embodiments, to obtain a solid plant ferment, the plant ferment concentrated under reduced pressure is spray-dried to remove the solvent, to obtain the solid plant ferment (also referred to as plant magnetic-field fermented powder).

In some embodiments, the plant ferment includes various active ingredients such as mannitol, gallic acid, dihydrocaffeic acid, syringin, 4-hydroxyphenolic acid, 3-phenyllactic acid, chlorogenic acid, caffeic acid, 1,5-di-o-caffroylquinic acid, 4-hyderoxy-3-phenlylactic acid methyl ester, calceolarioside B, plantainoside B, quercetin-3-glucuronide, cistanoside D, and luteolin.

In some embodiments, the plant ferment may be used for weight loss. Specifically, the plant ferment may increase an expression level of fat metabolism genes in cells and/or reduce an expression level of fat accumulation genes. The fat metabolism gene is selected from ATGL, LIPE, UCP1, UCP2, and a combination thereof, and the fat accumulation gene is PLN1 and/or PPARG2.

In some embodiments, the plant ferment prepared from the mulberry, pomegranate, purslane, wild bitter melon, and fennel can protect pancreatic β cells from denaturation, reduce lipid peroxidation, maintain the blood sugar of a subject constant, improve the ratio of high-density cholesterol to low-density cholesterol, reduce cholesterol and triglycerides, activate AMPK, a regulator of intracellular energy metabolism, and reduce blood sugar concentration, lipid concentration, and fat accumulation.

In some embodiments, the plant ferment may be further used to prepare a composition for weight loss. For example, the composition prepared from the plant ferment may be used to reduce the appetite of a subject, increase the time between meals of a subject, reduce hunger of a subject, and/or reduce the number of meals other than dinner of a subject per week. In addition, the composition may be used for reducing body weight, body mass index (BMI), whole body fat, and/or hip circumference of a subject.

In some embodiments, the composition prepared from the plant ferment may be used for reducing insulin resistance.

In some embodiments, the composition prepared from the plant ferment may be further prepared into a food composition, a dietary supplement composition, or a skin topical agent.

In some embodiments, the composition may be liquid (such as a plant fermented drink containing the plant ferment) or solid (such as a powder and a lozenge). In some embodiments, the plant ferment is used in a dose of 7 mL/day, and the solid composition is used in a dose of 0.58 g/day. In some embodiments, the composition is used in a dose of 7 g/day.

In some embodiments, the foregoing composition may be a medicament. In other words, the medicament includes an effective dose of plant ferment.

In some embodiments, the foregoing medicament may be manufactured into a dosage form suitable for enteral, parenteral, oral, or topical administration using techniques well known to those skilled in the art.

In some embodiments, the dosage form for enteral or oral administration includes, but is not limited to: a tablet, a troche, a lozenge, a pill, a capsule, a dispersible powder or granule, a solution, a suspension, an emulsion, a syrup, an elixir, a slurry, or other similar substances. In some embodiments, the dosage form for parenteral or topical administration includes, but is not limited to: an injection, a sterile powder, an external preparation, or other similar substances. In some embodiments, the administration manner of the injection may be subcutaneous injection, intraepidermal injection, intradermal injection, or intralesional injection.

In some embodiments, the foregoing medicament may include a pharmaceutically acceptable carrier widely used in drug manufacturing technology. In some embodiments, the pharmaceutically acceptable carrier may be one or more of the following carriers: a solvent, a buffer, an emulsifier, a suspending agent, a decomposer, a disintegrating agent, a dispersing agent, a binding agent, an excipient, a stabilizing agent, a chelating agent, a diluent, a gelling agent, a preservative, a wetting agent, a lubricant, an absorption delaying agent, a liposome, or other similar substances. The type and quantity of selected carriers fall within the scope of professionalism and routine technology of those skilled in the art. In some embodiments, the solvent of the pharmaceutically acceptable carrier may be water, normal saline, phosphate buffered saline (PBS), or aqueous solution containing alcohol.

In some embodiments, the foregoing medicament may be manufactured into an external preparation suitable for topical application to skin by using techniques well known to those skilled in the art, including but not limited to, an emulsion, a gel, an ointment, a cream, a patch, a liniment, a powder, an aerosol, a spray, a lotion, a serum, a paste, a foam, a drop, a suspension, a salve, and a bandage.

In some embodiments, the external preparation is prepared by mixing the medicament with a base well known to those skilled in the art. Specifically, the base may include one or more of the following additives: water, alcohols, glycol, hydrocarbons (such as petroleum jelly and white petrolatum), wax (such as paraffin and yellow wax), preserving agents, antioxidants, surfactants, absorption enhancers, stabilizing agents, gelling agents (such as Carbopol®974P, microcrystalline cellulose, and carboxymethyl cellulose), active agents, humectants, odor absorbers, fragrances. pH adjusting agents, chelating agents, emulsifiers, occlusive agents, emollients, thickeners, solubilizing agents, penetration enhancers, anti-irritants, colorants, propellants, etc. The selection and quantity of these additives fall within the scope of professionalism and routine technology of those skilled in the art.

In some embodiments, the foregoing composition may be a cosmeceutical. In other words, the cosmeceutical includes an effective dose of plant ferment. In addition, the cosmeceutical may also include an acceptable adjuvant widely used in cosmeceutical manufacturing technology. For example, the acceptable adjuvant may include one or more of the following reagents: a solvent, a gelling agent, an active agent, a preservative, an antioxidant, a screening agent, a chelating agent, a surfactant, a coloring agent, a thickening agent, a filler, a fragrance, and an odor absorber. The selection and quantity of these reagents fall within the scope of professionalism and routine techniques of those skilled in the art.

In some embodiments, the cosmeceutical may be made into a form suitable for skincare or makeup by using techniques well known to those skilled in the art, including but not limited to: an aqueous solution, an aqueous-alcohol solution or an oily solution, and an emulsion, a gel, an ointment, a cream, a mask, a patch, a pack, a liniment, a powder, an aerosol, a spray, a lotion, a serum, a paste, a foam, a dispersion, a drop, a mousse, a sunblock, a tonic water, a foundation, a makeup remover product, a soap, and other body cleansing products that are an oil-in-water type, a water-in-oil type, or a composite type.

In some embodiments, the cosmeceutical may also be used in combination with one or more of the following external use agents with known activity: whitening agents (such as tretinoin, catechin, kojic acid, arbutin, and vitamin C), humectants, anti-inflammatory agents, bactericides, ultraviolet absorbers, plant extracts (such as aloe extracts), skin nutrients, anesthetics, anti-acne agents, antipruritics, analgesics, antidermatitis agents, antihyperkeratolytic agents, anti-dry skin agents, antipsoriatic agents, antiaging agents, antiwrinkle agents, antiseborrheic agents, wound-healing agents, corticosteroids, and hormones. The selection and quantity of these external use agents fall within the scope of professionalism and routine technology of those skilled in the art.

In some embodiments, the foregoing composition may be an edible food. In other words, the edible product includes a specific content of plant ferment. In some embodiments, the edible product may be a general food, a food for special health use (FoSHU), or a dietary supplement.

In some embodiments, the foregoing edible product may be manufactured into a dosage form suitable for oral administration using techniques well known to those skilled in the art. In some embodiments, the foregoing general food may be the edible product itself. In some embodiments, the general food may be, but is not limited to: beverages, fermented foods, bakery products, or condiments.

In some embodiments, the plant ferment obtained may further used as a food additive to prepare a food composition containing the Chinese medicine ferment. Herein, the Chinese medicine ferment of any embodiment can be added during the preparation of raw materials by conventional methods, or the plant ferment of any embodiment can be added during food making, to prepare an edible product (that is, a food composition) for humans and non-human animals with any edible material.

### Example 1: Preparation of Plant Ferment

First, a mulberry juice (origin in Taiwan), a pomegranate juice (origin in Taiwan), purslane leaves (origin in China), a wild bitter melon fruit (origin in China), a fennel fruit (origin in China), and water were mixed in a weight ratio of 2:6:2:1:2:90, 3% of glucose solution was added, and sterilization and extraction were simultaneously carried out at 95° C. for 1 h, to obtain a plant extract. Next, the plant extract was cooled down to room temperature for subsequent three-stage fermentation.

0.1% (w/w) of yeast (Saccharomyces cerevisiae, purchased from the Bioresource Collection and Research Center, Taiwan, with a deposit number of BCRC20271) was inoculated into the plant extract to ferment at 28±5° C. for 1 day to obtain a first initial fermentation broth. Then, 0.05% (w/w) of Lactobacillus plantarum TC1378 (deposited in the Bioresource Collection and Research Center, Taiwan, with a Taiwan deposit number of BCRC910760 and an international deposit number of DSM32451) was inoculated into the first initial fermentation broth to ferment at 28±5° C. for 1 day to obtain a second initial fermentation broth. Then, 5% (w/w) of Acetobacter aceti (purchased from the Bioresource Collection and Research Center, Taiwan, with a deposit number of BCRC11688) was inoculated into the second initial fermentation broth to ferment at 28±5° C. for 5 days to obtain a third initial fermentation broth. Herein, without removing the three bacteria, the third initial fermentation broth obtained has a sugar content of about 4.7° Bx, a pH value of about 3.5, and about 5% of alcohol.

The third initial fermentation broth was filtered by a 200-mesh filter, and concentrated under reduced pressure at 60±5° C., to obtain a plant fermented stock solution containing about 0.5% of alcohol. Water was added into the plant fermented stock solution to supplement the weight reduced through the concentration under reduced pressure, and the resulting plant fermented stock solution was heated for sterilization at 95° C. for 90 min, to obtain a plant ferment.

### Example 2: Preparation of Plant Aqueous Extract

A mulberry juice (origin in Taiwan), a pomegranate juice (origin in Taiwan), purslane leaves (origin in China), a wild bitter melon fruit (origin in China), a fennel fruit (origin in China), and water were mixed in a weight ratio of 2:6:2:1:2:90, 3% of glucose solution was added, and sterilization and extraction were simultaneously carried out at 95° C. for 1 h, to obtain a plant aqueous extract. Herein, the plant aqueous extract has a pH value of 6.3.

### Example 3: Analysis on Cell Experiment of Fat-Related Genes

Herein, the expression level of fat metabolism genes and fat accumulation genes in mouse bone marrow stromal cells (purchased from BCRC with a number of 6566; hereinafter referred to as OP9 cells) treated with the plant ferment prepared in Example 1 was determined by using the RNA extraction kit, SuperScript® III Reverse Transcriptase, KAPA SYBR®FAST qPCR reagent kit, and quantitative PCR machine. In addition, the fat metabolism genes include the ATGL gene (Gene ID: 57104), LPE (HSL) gene (Gene ID: 3991), UCP1 gene (Gene ID: 7350), and LCP2 gene (Gene ID: 7351); and the fat accumulation genes include the PLIN1 (Gene ID: 5346) and PPARG2 gene (Gene ID: 5468).

The protein encoded by the ATGL gene can promote the hydrolysis of triglycerides, the first step of fat metabolism, in lipid droplet cells. The protein encoded by the LIPE gene can hydrolyze triglycerides into free fatty acids and is responsible for the conversion of cholesteryl ester to free cholesterol. The protein encoded by the UCP1 gene and UCP2 gene contributes to the conversion of fat cells and promotes fat combustion. The protein encoded by the PLIN1 gene can regulate fat metabolism in lipid droplet cells, where a low expression level of the PLIN1 gene promotes fat metabolism in cells. The protein encoded by the PPARG gene can regulate the beta oxidation process of fatty acids to regulate fat metabolism.

Cell culture medium used was the α-minimum essential medium (α-MEM, purchased from Gibco, number 12000-022) containing 20% of fetal bovine serum (FBS, purchased from Gibco, number 10438-026, USA) and 1% of antibiotic-antimycotic (purchased from Gibco, number 15240-062).

First, the OP9 cells were inoculated into a 6-well culture plate containing 2 mL of culture medium in a density of 1×10⁵ cells per well, and cultured at 37° C. for 24 h. Next, the OP9 cells were divided into three groups: a blank group, a control group, and an experimental group. Then, the cell culture medium in each group was replaced with 2 mL of experimental culture medium per well, and then cultured at 37° C. for 12 h. The experimental culture medium in the blank group was a cell culture medium with no treatment (that is, no additional compounds or plant ferment was added into the cell culture medium). The experimental culture medium in the control group was a cell culture medium containing 0.025 mg/mL of plant aqueous extract obtained in Example 2. The experimental culture medium in the experimental group was a cell culture medium containing 0.025 mg/mL of plant ferment obtained in Example 1. Each group was repeated for three times.

The cell membranes of the OP9 cells treated in each group were broken with a cell lysis buffer to form a cell solution. Next, RNA of the three groups of cell solutions was extracted separately by using an RNA extraction reagent kit (purchased from Geneaid, Taiwan, Lot No. FC24015-G). Then, 1000 ng of the extracted RNA in each group was used as a template, and the extracted RNA was reverse transcribed into corresponding cDNA by the SuperScript® III reverse transcriptase (purchased from Invitrogene, USA, number 18080-051). Subsequently, the quantitative real-time reverse transcription polymerase chain reaction was carried out on the three groups of cDNA with the primers (SEQ ID NO: 1 to SEQ ID NO: 14) in Table 1 by using the ABI StepOnePlus™ Real-Time PCR system (Thermo Fisher Scientific, USA) and the KAPA SYBR FAST (purchased from Sigma, USA, number 38220000000) to observe the expression level of the ATGL gene, LIPE (HSL) gene, UCP1 gene, UCP2 gene, PLIN1 gene, and PPARG2 gene in the OP9 cells in the three groups. The instrument setting conditions for the quantitative real-time reverse transcription polymerase chain reaction were 95° C. for 20 s, 95° C. for 3 s, 60° C. for 30 s, a total of 40 cycles, and gene quantification was carried out by the 2^{-ΔCt} method (using the m-ACTB gene as an internal reference gene). Herein, the quantitative real-time reverse transcription polymerase chain reaction with cDNA can indirectly quantify the mRNA expression level of the ATGL gene, LIPE (HSL) gene, UCP1 gene, UCP2 gene, PLIN1 gene, and PPARG2 gene, and then infer the expression level of the protein encoded by the ATGL gene, LIPE (HSL) gene, UCP1 genes, UCP2 gene, PLIN1 gene, and PPARG2 gene.

TABLE 1

| | Sequence | |
|---|---|---|
| Primer name | number | Primer sequence |
| ATGL-F | SEQ ID NO: 1 | GGATGGCGGCATTTCAGACA |
| ATGL-R | SEQ ID NO: 2 | CAAAGGGTTGGGTTGGTTCAG |
| LIPE (HSL)-F | SEQ ID NO: 3 | TGGCACACCATTTTGACCTG |
| LIPE (HSL)-R | SEQ ID NO: 4 | TTGCGGTTAGAAGCCACATAG |
| UCP1-F | SEQ ID NO: 5 | AGGCTTCCAGTACCATTAGGT |
| UCP1-R | SEQ ID NO: 6 | CTGAGTGAGGCAAAGCTGATTT |
| UCP2-F | SEQ ID NO: 7 | ATGGTTGGTTTCAAGGCCACA |
| UCP2-R | SEQ ID NO: 8 | CGGTATCCAGAGGGAAAGTGAT |
| PLIN1-F | SEQ ID NO: 9 | GGGACCTGTGAGTGCTTCC |
| PLIN1-R | SEQ ID NO: 10 | GTATTGAAGAGCCGGGATCTTTT |
| PPARG2-F | SEQ ID NO: 11 | TCGCTGATGCACTGCCTATG |
| PPARG2-R | SEQ ID NO: 12 | GAGAGGTCCACAGAGCTGATT |
| m-ACTB-F | SEQ ID NO: 13 | GGCTGTATTCCCCTCCATCG |
| m-ACTB-R | SEQ ID NO: 14 | CCAGTTGGTAACAATGCCATGT |

R represents REVERSE, and F represents FORWARD.

It is to be noted that the relative gene expression of the ATGL gene, LIPE (HSL) gene, UCP1 gene, UCP2 gene, PLIN1 gene, and PPARG2 gene shown in figures was presented by relative magnification. The standard deviation was calculated by using the STDEV formula of Excel software, and whether there was a statistically significant difference was analyzed by one-tailed student t-test in Excel software. In the figures, “*” represents a p value less than 0.05, “**” represents a p value less than 0.01, and “***” represents a p value less than 0.001. More “*” represents more significant statistical differences.

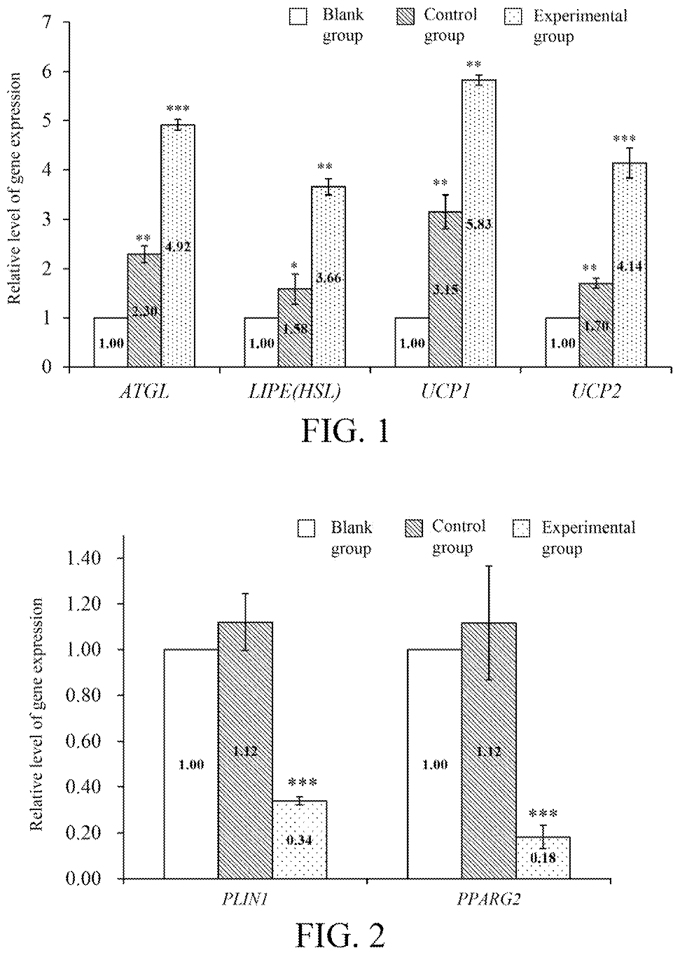

Referring to FIG. 1, when the expression level of each of the ATGL gene, LIPE (HSL) gene, UCP1 gene, and UCP2 gene in the blank group was regarded as 1 (that is, 100%), for the control group relative to the blank group, an expression level of the ATGL gene was 2.30 (that is, 230%), an expression level of the LPE (HSL) gene was 1.58 (that is, 158%), an expression level of the UCP1 gene was 3.15 (that is, 315%), and an expression level of the UCP2 gene was 1.70 (that is, 170%); and for the experimental group relative to the blank group, an expression level of the ATGL gene was 4.92 (that is, 492%), an expression level of the LIPE (HSL) gene was 3.66 (that is, 366%), an expression level of the UCP1 gene was 5.83 (that is, 583%), and an expression level of the UCP2 gene was 4.14 (that is, 414%). It can be learned that, compared with the blank group and the control group, the ATGL gene, LIPE (HSL) gene, UCP1 gene, and UCP2 gene of the experimental group were all significantly increased. In other words, the plant ferment increase the expression level of fat metabolism genes in OP9 cells, indicating that the plant ferment can promote lipolysis.

Referring to FIG. 2, when the expression level of each of the PLIN1 gene and PPARG2 gene in the blank group was regarded as 1 (that is, 100%), for the control group relative to the blank group, an expression level of the PLIN1 gene was 1.12 (that is, 112%), and an expression level of the PPARG2 gene was 1.12 (that is, 112%); and for the experimental group relative to the blank group, an expression level of the PLIN1 gene was 0.34 (that is, 34%), and an expression level of the PPARG2 gene was 0.18 (that is, 18%). It can be learned that, compared with the blank group and the control group, the PLIN1 gene and PPARG2 gene of the experimental group were all significantly reduced. In other words, the plant ferment reduce the expression level of fat accumulation genes in OP9 cells, indicating that the plant ferment can reduce fat accumulation.

### Example 4: Analysis on Experiment of Lipid Droplet Accumulation

Fat loss refers to lipolysis. The lipolysis refers to the process in which triglyceride (TG) stored in fat cells is gradually degraded into fatty acid (FA) and glycerol. Herein, the content of glycerol in fat cells is used as a quantitative indicator to observe whether there is lipolysis.

Cell culture medium used was the α-minimum essential medium (α-MEM, Gibco, number 12000-022) containing 20% of fetal bovine serum (FBS, Gibco, number 10438-026) and 1% of antibiotic-antimycotic (Gibco, number 15240-062).

First, 8×10⁴ OP9 cells (purchased from the American Type Culture Collection, ATCC®, number ATCC CRL-2749) were inoculated into a 24-well culture plate containing 500 μL of cell culture medium per well, and cultured at 37° C. for 7 days. During the 7-day cell culture, the cell culture medium was changed every 3 days. After the 7 days of culture, the formation of lipid droplets in the OP9 cells was observed by using a microscope (ZEISS; magnification 400×) to confirm that the OP9 cells were fully differentiated into fat cells for subsequent experiments.

Then, the differentiated fat cells were divided into an experimental group, a control group, and a blank group. The cell culture medium in each group was replaced with 500 μL of experimental medium per well, and then cultured at 37° C. for 7 days. During the 7-day culture, the experimental medium was replaced with a fresh 500 PL one every 3 days. The experimental culture medium in the experimental group was a cell culture medium containing 0.025 mg/mL of plant ferment obtained in Example 1. The experimental culture medium in the control group was a cell culture medium containing 0.025 mg/mL of plant aqueous extract obtained in Example 2. The experimental culture medium in the blank group was just a cell culture medium (without the plant ferment).

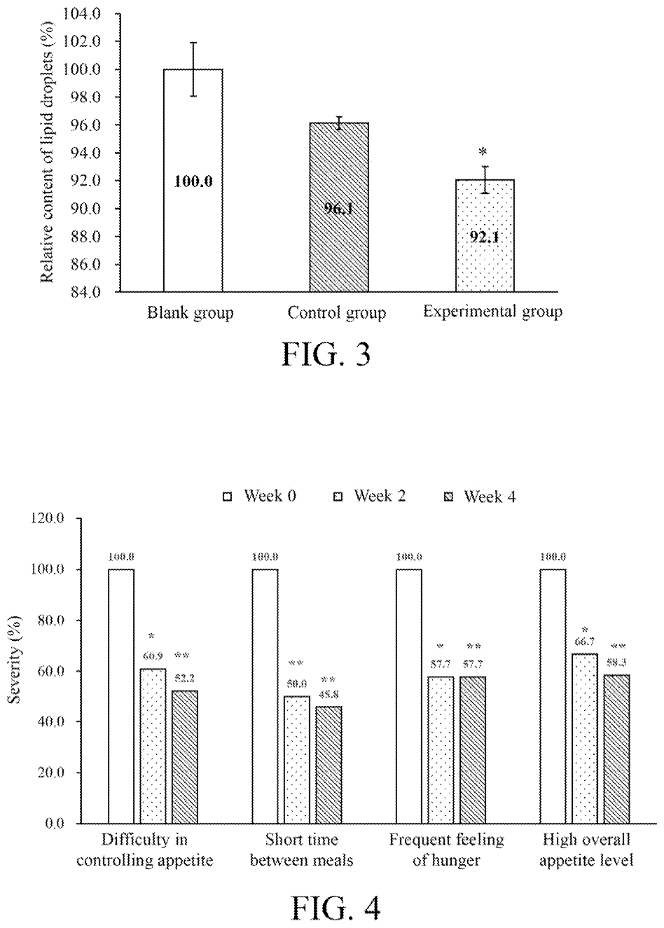

The content of glycerol was determined by using a glycerol cell-based assay kit (purchased from Cayman, USA, product number 10011725) according to the following steps. The experimental culture medium (that is, the experimental culture medium in which fat cells had been cultured, without the fat cells) in each group was collected, 25 μL of the experimental culture medium was transferred into a new 96-well culture plate, 100 μL of reconstituted free glycerol assay reagent was added into each well to react at room temperature for 15 min, and the absorbance OD_{540 nm} of each group was read from the 96-well culture plate by an ELISA reader, to quantify the content of glycerol released into the experimental culture medium through lipolysis, as shown in FIG. 3. Herein, the content of glycerol was directly proportional to the amount of lipolysis. The obtained results were analyzed by student t-test using Excel software to determine whether there was a statistically significant difference between two sample groups. (In the figures, “*” represents a p value less than 0.05, “**” represents a p value less than 0.01, and “***” represents a p value less than 0.001. More “*” represents more significant statistical differences.)

Refer to FIG. 3. Herein, a lipid droplet content of the blank group was regarded as 100%. A lipid droplet content of the control group was 96.1%, and a lipid droplet content of the experimental group was 92.1%. Therefore, compared with the blank group and the control group, the lipid droplet content in fat cells of the experimental group was significantly reduced. It can be learned that the plant ferment effectively promote lipolysis, and has the function of improving fat metabolism of subjects, thereby achieving fat loss.

Based on this, the plant ferment effectively inhibits fat accumulation, and has the function of reducing fat formation of subjects, thereby achieving fat loss. In addition, as compared with the plant aqueous extract, the plant ferment prepared by microbial fermentation has a better effect of reducing the lipid droplet content.

### Example 5: Human Subject Experiment

7 subjects (with a body fat percentage greater than 25% or a BMI greater than 24) were allowed to drink a 50 mL plant ferment drink (containing 7 g of plant ferment obtained in Example 1 with water added to a total volume of 50 mL) every day for 4 weeks.

### Example 5-1: Human Subject Experiment—Questionnaire Analysis

Test method: The 7 subjects were subjected to the fill-out of a questionnaire about various conditions related to diet before drinking (week 0), after drinking for 2 weeks (week 2), and after drinking for 4 weeks (week 4). The survey and scoring method was shown in Table 2. In addition, at week 2 and week 4, the 7 subjects were surveyed for the satisfaction on the appetite suppressant effect of the plant ferment.

TABLE 2

| Whether the symptoms occurred | 1 | 2 | 3 | 4 | 5 |
|---|---|---|---|---|---|
| Question 1. Difficulty in | | | | | |
| controlling appetite | | | | | |
| Question 2. Short time | | | | | |
| between meals | | | | | |
| Question 3. Frequent | | | | | |
| feeling of hunger | | | | | |
| Question 4. High overall | | | | | |
| appetite level | | | | | |

Survey on gastrointestinal conditions during testing
(only one answer can be chosen)

| | | | | | |
|---|---|---|---|---|---|
| 1. Do you have a habit | [symbol] | [symbol] | [symbol] | [symbol] | [symbol] |
| of eating a late-night meal? | No | 1-2 | 3-4 | 5-6 | Every |
| 2. Do you have a habit | | times a | times a | times a | day |
| of eating snack? | | week | week | week | |
| 3. How many meals do | [symbol] | [symbol] | [symbol] | [symbol] | — |
| you usually eat in a day? | One | Two | Three | Four | |

In Table 2, for Question 1 to Question 4, 1 represents complete disagreement, 2 represents disagreement, 3 represents fair, 4 represents agreement, and 5 represents complete agreement. In addition, the average number of meals other than dinner of the 7 subjects per week may be sorted out by conducting a survey on the gastrointestinal conditions during testing.

As shown in FIG. 4 to FIG. 7, the statistical significance difference among measurement results at week 0, week 2, and week 4 was counted and analyzed through student t-test. In FIG. 4 to FIG. 6, “*” represents a p value less than 0.05 in comparison with week 0, and “*” represents a p value less than 0.01 in comparison with blank group.

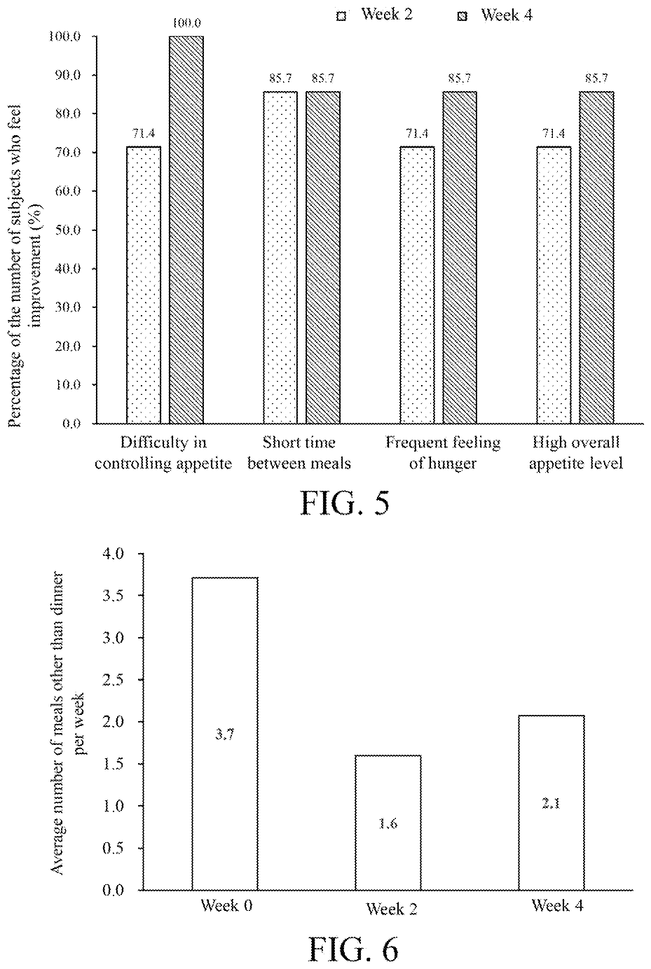

Referring to FIG. 4 and FIG. 5, the occurrence of symptoms (that is, difficulty in controlling appetite, short time between meals, frequent feeling of hunger, and high overall appetite level) before drinking (week 0) was regarded as 100%. After drinking the plant ferment drink for 2 weeks, the condition that the 7 subjects have difficulty in controlling appetite was significantly reduced to about 60.9% (there were 5 subjects who feel improvement, accounting for about 71.4%), the condition that the 7 subjects have a short time between meals was significantly reduced to about 50.0% (there were 6 subjects who feel improvement, accounting for about 85.7%), the condition that the 7 subjects often feel hungry was significantly reduced to about 57.7% (there were 5 subjects who feel improvement, accounting for about 71.4%), and the condition that the 7 subjects have a high overall appetite level was significantly reduced to about 66.7% (there were 5 subjects who feel improvement, accounting for about 71.4%). After drinking the plant ferment drink for 4 weeks, the condition that the 7 subjects have difficulty in controlling appetite was significantly reduced to about 52.2% (there were 7 subjects who feel improvement, accounting for about 100%), the condition that the 7 subjects have a short time between meals was significantly reduced to about 45.8% (there were 6 subjects who feel improvement, accounting for about 85.7%), the condition that the 7 subjects often feel hungry was significantly reduced to about 57.7% (there were 6 subjects who feel improvement, accounting for about 85.7%), and the condition that the 7 subjects have a high overall appetite level was significantly reduced to about 58.3% (there were 6 subjects who feel improvement, accounting for about 85.7%).

It can be learned that drinking of the plant ferment drink containing the plant ferment for at least 2 weeks reduces the appetite, increases the time between meals, and/or reduces hunger.

Referring to FIG. 6, compared with the average number 3.7 of meals other than dinner per week of the 7 subjects before drinking (week 0), the average number of meals other than dinner per week of the 7 subjects after drinking the plant ferment drink for 2 weeks was significantly reduced to 1.6, and the average number of meals other than dinner per week of the 7 subjects after drinking the plant ferment drink for 4 weeks was significantly reduced to 2.1. In addition, there were 5 of the 7 subjects who feel improvement, accounting for about 71.4%. It can be learned that drinking of the plant ferment drink containing the plant ferment for at least 2 weeks reduce the number of meals other than dinner per week of a subject.

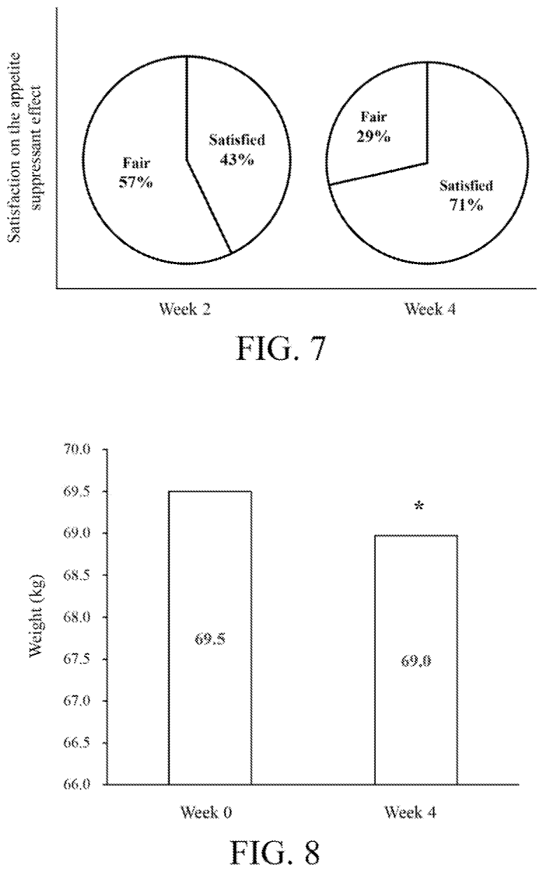

Moreover, the satisfaction evaluation on the appetite suppressant effect was carried out on the 7 subjects after drinking the plant ferment drink for 2 weeks and 4 weeks. Referring to FIG. 7, after drinking the plant ferment drink for 2 weeks, 4 of the 7 subjects feel “fair” (57% of the total) and 3 of the 7 subjects feel “satisfied” (43% of the total) with the appetite suppressant effect; and after drinking the plant ferment drink for 4 weeks, the number of subjects who feel “fair” with the appetite suppressant effect decreases to 2 (29% of the total), while the number of subjects who feel “satisfied” with the appetite suppressant effect increases to 5 (71% of the total). It can be learned that drinking of the plant ferment drink containing the plant ferment significantly increases the feeling of appetite suppression of a subject.

### Example 5-2: Human Subject Experiment—Body Composition Analysis

Test method: The 7 subjects were subjected to the measurement of weight, whole body fat percentage, and trunk fat percentage using a scale (brand: TANITA, product: limb and torso body composition meter, model: BC-545F) and the measurement of waist circumference using a cloth ruler before drinking (week 0) and after drinking for 4 weeks (week 4). During the measurement of waist circumference, it was necessary to remove the clothing covering the subject's waist, and when the subject stands relaxed with hands hang down naturally, the waist circumference of the subject was measured by using the subject's navel as the horizontal measurement point.

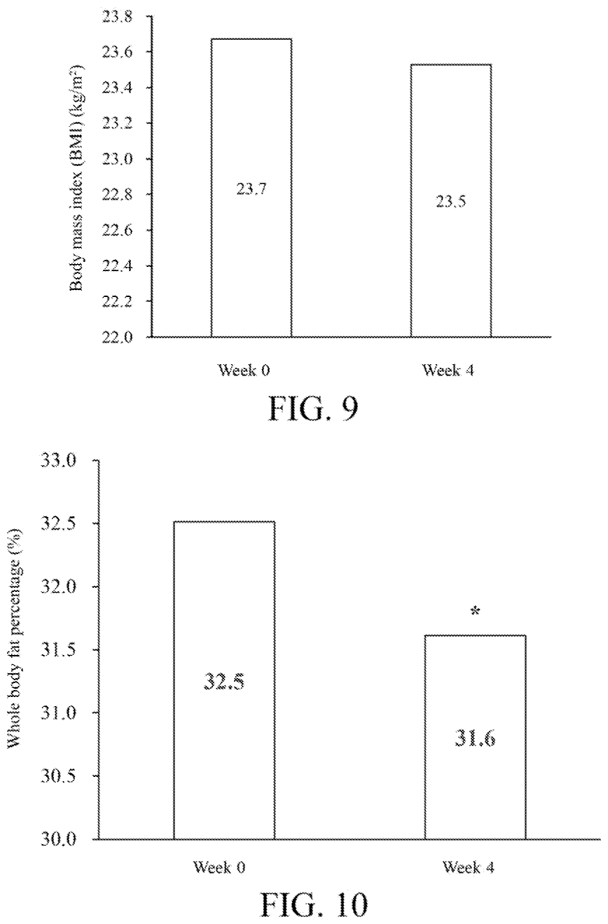

Referring to FIG. 8 and FIG. 9, compared with the average weight 69.5 kg and the average BMI 23.7 before drinking (week 0), for the subjects after drinking the plant ferment drink for 4 weeks, the average weight was significantly reduced by 0.5 kg (to 69.0 kg), and the average BMI was reduced by 0.2 (to 23.5). In addition, there were 6 of the 7 subjects improved (accounting for about 85.7%).

Referring to FIG. 10, compared with the average whole body fat percentage 32.5% before drinking (week 0), the average whole body fat percentage of the subjects after drinking the plant ferment drink for 4 weeks was significantly reduced by about 0.9% (to 31.6%).

Referring to FIG. 11, compared with the average hip circumference 100.9 cm before drinking (week 0), the average hip circumference of the subjects after drinking the plant ferment drink for 4 weeks was significantly reduced by about 1 cm (to 99.9 cm).

It can be learned that drinking of the plant ferment drink containing the plant ferment for at least 4 weeks reduce the weight, BMI, whole body fat percentage, and hip circumference of a subject, thereby achieving weight loss.

### Example 5-3: Human Subject Experiment—Insulin Resistance Index Analysis

Test method: The 7 subjects were subjected to the blood collection and the measurement of insulin resistance index in the blood entrusted to LEZEN Lab. (Taiwan) before drinking (week 0) and after drinking for 4 weeks (week 4), to detect the change of insulin resistance in the blood before and after drinking the plant ferment drink. Insulin resistance refers to the phenomenon that since the sensitivity of human cells (especially liver, muscle, and fat cells) to insulin is reduced, blood glucose cannot smoothly enter the cells for breakdown to provide energy; and in response to this, the body allows the pancreas to secrete more insulin, resulting in high concentrations of insulin in the patient's blood. A lower insulin resistance indicates a higher sensitivity of the cells to insulin.

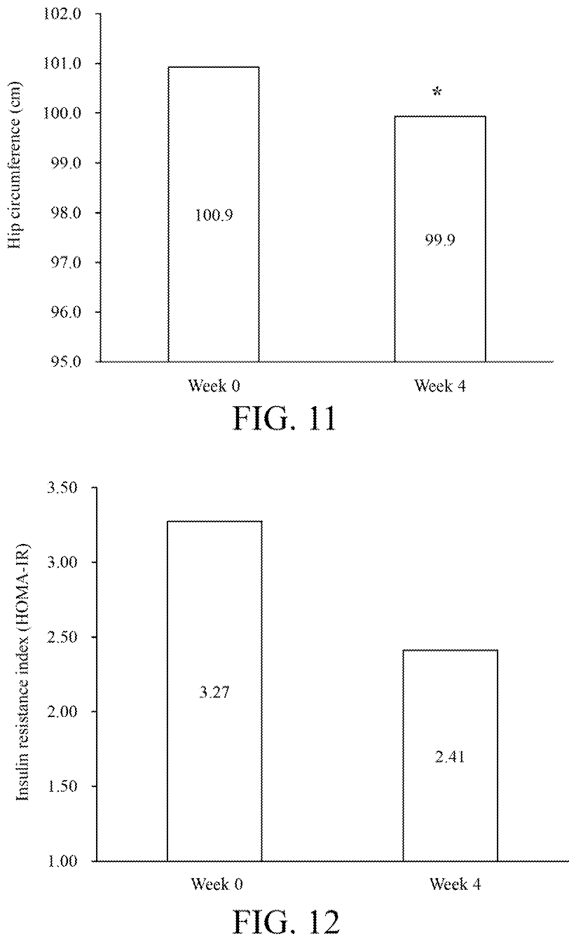

Referring to FIG. 12, compared with the average insulin resistance index 3.27 of the subjects before drinking (week 0), the average insulin resistance index of the subjects after drinking the plant ferment drink for 4 weeks can be significantly reduced to 2.41 (to 74%).

It can be learned that drinking of the plant ferment drink containing the plant ferment for at least 4 weeks can reduce the insulin resistance index of a subject, indicating that the sensitivity of body cells of the subject to glucose was increased, so that excess energy was less likely to be stored as fat, to reduce fat accumulation.

### Example 6: HPLC Fingerprints of Plant Ferment and Plant Aqueous Extract

Quantitative and qualitative analysis was carried out on biologically active substances in the plant ferment prepared in Example 1 and the plant aqueous extract prepared in Example 2 by high performance liquid chromatography (HPLC).

The solvents used were methanol and water with 0.1% formic acid added each, with a flow rate set to 1 mL/min and an elution condition set to methanol: water of 2:98 at 0 min, methanol:water of 2:98 at 10 min, methanol:water of 70:30 at 40 min, methanol:water of 100:0 at 50 min, and methanol:water of 100:0 at 60 min. In addition, the plant ferment and the plant aqueous extract each has a sample concentration of 50 mg/mL and an injection volume for analysis of 10 μL. Herein, during the experiment, the column temperature was set to 40° C.

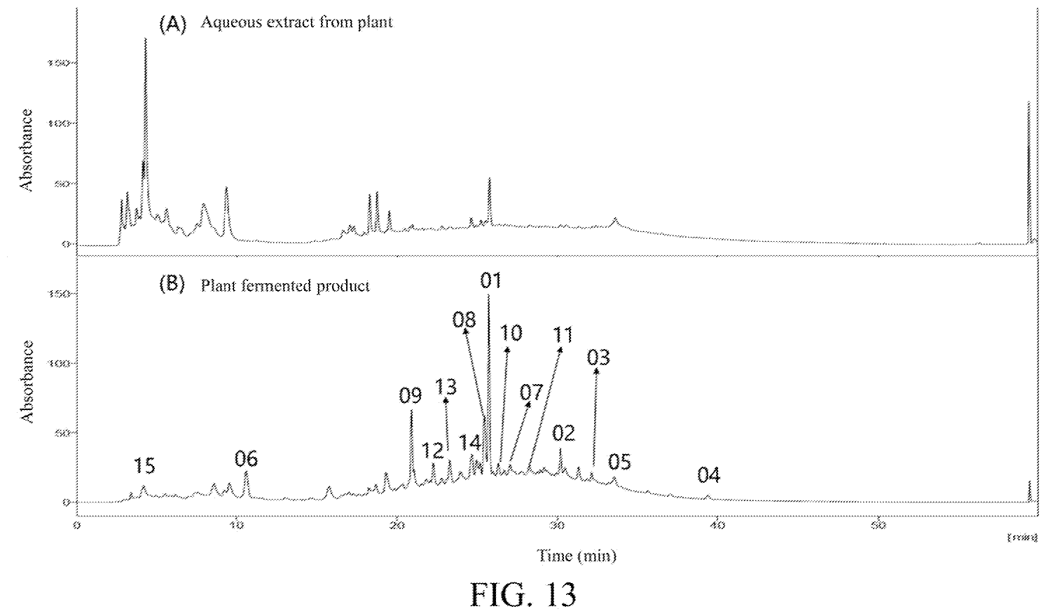

Refer to FIG. 13. On top (A) of the figure is the fingerprint of the plant aqueous extract prepared in Example 2. It can be learned that most of the peaks of biologically active substances in the plant aqueous extract were resolved before 10 minutes, and the peaks of a few of biologically active substances were resolved at about 15-20 minutes, 25 minutes, and 34-35 minutes. Compared with the fingerprint of the plant aqueous extract, on bottom (B) of the figure is the fingerprint of the plant ferment prepared in Example 1. It can be learned that most of the peaks of biologically active substances in the plant ferment were resolved at about 20-40 minutes, and other peaks were resolved at about 5 minutes, 10 minutes, and 40 minutes, etc.

Specifically, in the fingerprint of the plant ferment prepared in Example 1, the peak of the biologically active substance TCI-LFT-15 (marked as 15) was resolved at about minutes, the peak of the biologically active substance TCI-LET-06 (marked as 06) was resolved at about 11 minutes, the peaks of the biologically active substances TCI-LFT-09 (marked as 09), TCI-LFT-112 (marked as 12), TCI-LFT-13 (marked as 13), TCI-LFT-14 (marked as 14), TCI-LFT-08 (marked as 08), TCI-LFT-01 (marked as 01), TCI-LFT-10 (marked as 10), TCI-LFT-07 (marked as 07), TCI-LFT-11 (marked as 11), and TCI-LFT-02 (marked as 02) (in chronological order) were resolved at 20-30 minutes, and the peaks of the biologically active substances TCI-LFT-03 (marked as 03), TCI-LFT-05 (marked as 05), and TCI-LFT-04 (marked as 4) were resolved at 30-40 minutes.

It can be learned that the plant aqueous extract and the plant ferment have different fingerprints and different contents of biologically active substances.

### Example 7: Analysis and Test of Biologically Active Substances in Plant Ferment

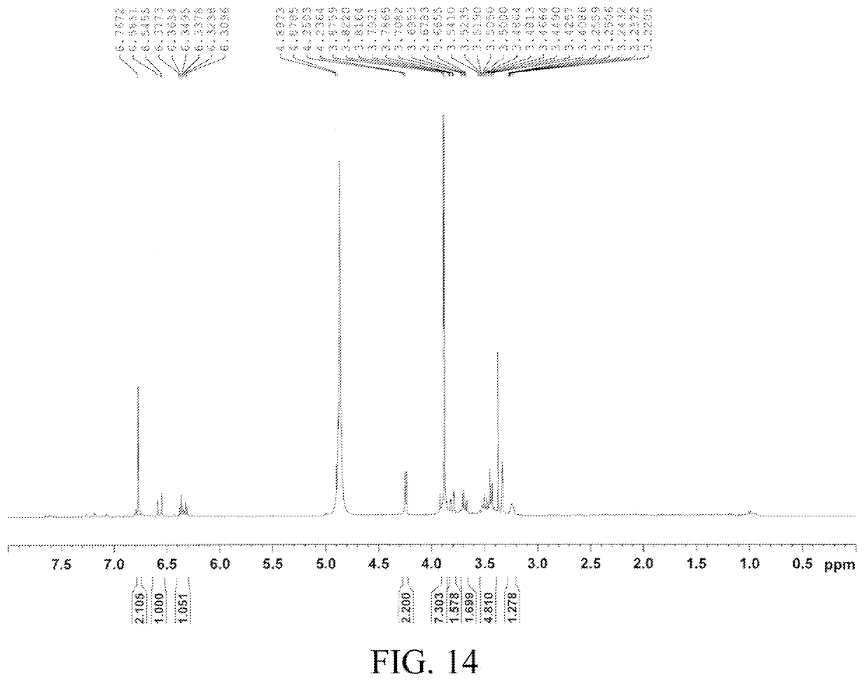

For isolation and purification of biologically active substances in the plant ferment, the isolation was carried out by bioassay guided fractionation to obtain compounds TCI-LFT-01 to TCI-LFT-15, and these compounds isolated were identified by using a nuclear magnetic resonance spectrometer. Specific spectra are shown in FIG. 14 to FIG. 29.

The equipment, setting methods, and equipment sources used in the analysis and test of biologically active substances of the plant ferment are described as follows:

(1) Nuclear magnetic resonance (NMR) spectrometer. 1D and 2D spectra are obtained by using the Ascend 400 MHz spectrometer, Bruker Co., Germany. δ is used to represent a chemical shift in units of ppm.

(2) Mass spectrometer (MS): tandem mass spectrometry-two-dimensional ion trap tandem Fourier-transform mass spectrometry and ESI-MS/MS is determined by using the Bruker amaZon SL system in units of m/z.

(3) Medium pressure liquid chromatograph (MPLC): CombiFlash® Rf⁺, Teledyne ISCO, Lincoln, Nebr.; high performance liquid chromatograph (HPLC): the HPLC pertains to the Agilent 1200 series, where a degasser is the Agilent 1322A vacuum degasser, an elution solvent is delivered by using the Agilent G1311A quaternary pump; a multiple wavelength detector (MWD) is Agilent G1314B; and a diode army detector (DAD) is Agilent 1260 Infinity DAD VL G1315D with detection wavelengths of 210 nm, 280 nm, 320 nm, and 365 nm. (Agilent Germany).

(4) Analytical column: Luna® 5 μm C18(2) 100 Å (250×10 mm, Phenomenex, USA).

(5) Column chromatography packing materials: Sephadex LH-20 (Pharmacia, Piscataway, N.J., USA). Diaion HP-20 (Mitsubishi Chemical Co., Japan) Merck Kieselgel 60 (40-63 μm, Art. 9385) Merck LiChroprep® RP-18 (40-63 μm, Art. 0250).

(6) Thin-layer chromatography: TLC aluminium sheets (Silica gel 60 F254, 0.25 mm, Merck, Germany) and TLC aluminium sheets (RP-18 F254-S, 0.25 mm, Merck, Germany).

(7) UV lamp: UVP UVGL-25 with a wavelength of 254 nm and 365 nm.

(8) Solvents and sources thereof: n-hexane, ethyl acetate, acetone, methanol, ethanol, acetonitrile (commercially available from Merck & Co., Taiwan), chloroform-d1 (with a deuteration degree of 99.5%), methanol-d4 (with a deuteration degree of 99.5%), deuterium oxide (with a deuteration degree of >99.8%), and dimethyl sulfoxide-d6 (with a deuteration degree of >99.9%) (commercially available from Merck & Co., Taiwan).

First, 10 L of plant ferment prepared in Example 1 was separated through liquid-liquid extraction with n-butanol and water in equal proportions to obtain 22.6 g of n-butanol-soluble fraction (BuF) and 196.3 g of water-soluble fraction (WF).

Next, 100 g of WF was preliminarily separated by column chromatography on the macroporous Dianion HP-20 resin with pure water, pure water-methanol (with a volume ratio of 1:1), and methanol as eluants in sequence, to obtain 3 water fractions (WF1 to WF3).

In addition, the WF1 and WF2 each was separated by using a reverse-phase medium-pressure liquid chromatograph (RP-MPLC) to obtain a plurality of WF1 eluted substances and a plurality of WF2 eluted substances. The elution used was linear elution from water to methanol with an elution time of 60 min and a flow rate of 10 mL/min. Then, the plurality of WF1 eluted substances and the plurality of WF2 eluted substances were chromatographed by thin-layer chromatography (TLC aluminium sheets, Silica gel 60 F254, 0.25 mm, Merck, Germany), and the WF1 eluted substances and WF2 eluted substances with similar results were combined, to obtain 3 WF1 sub-fractions (WF1-1 to WF1-3) and 4 WF2 sub-fractions (WF2-1 to WF2-4).

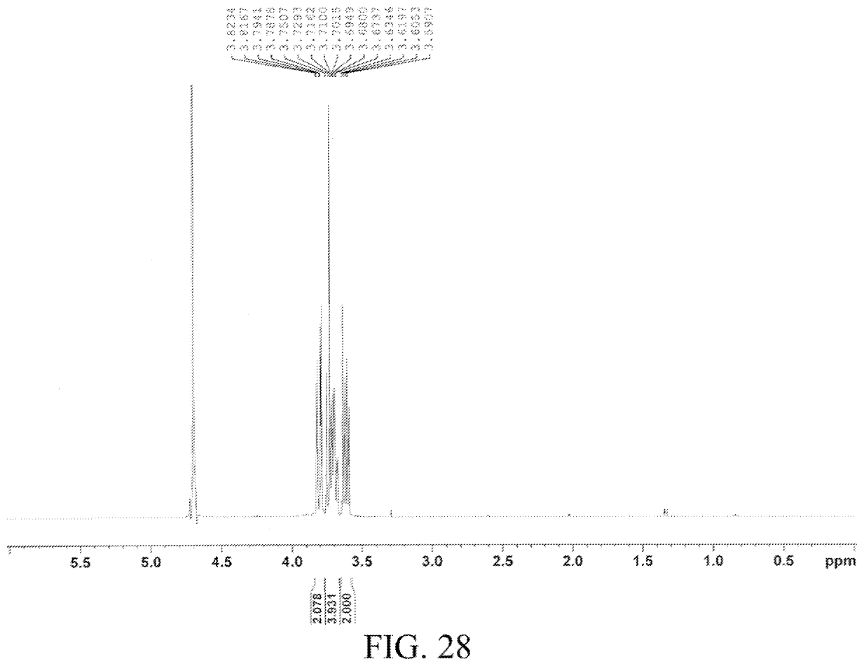

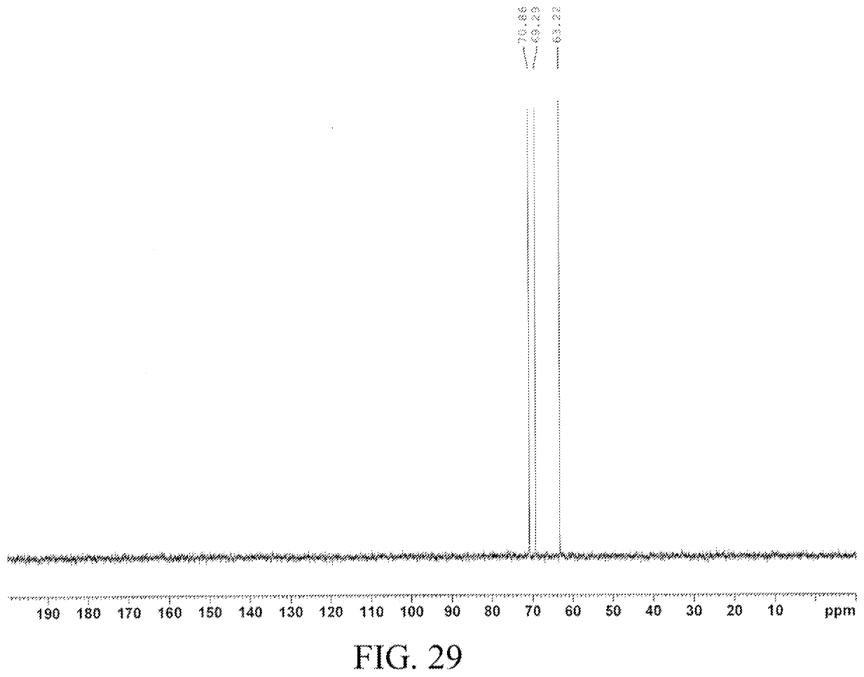

The WF1-1 was purified by normal-phase silica gel column chromatography (with a solvent of ethyl acetate and methanol in a volume ratio of 1:1) to obtain 3.7 mg of compound TCI-LTF-15. After the chemical structure of TCI-LTF-15 was analyzed through ¹H-NMR and ¹³C-NMR, TCI-LTF-15 was determined as mannitol with a molecular mass of 182. Its ¹H-NMR spectrum is shown in FIG. 28, and its ¹³C-NMR spectrum is shown in FIG. 29.

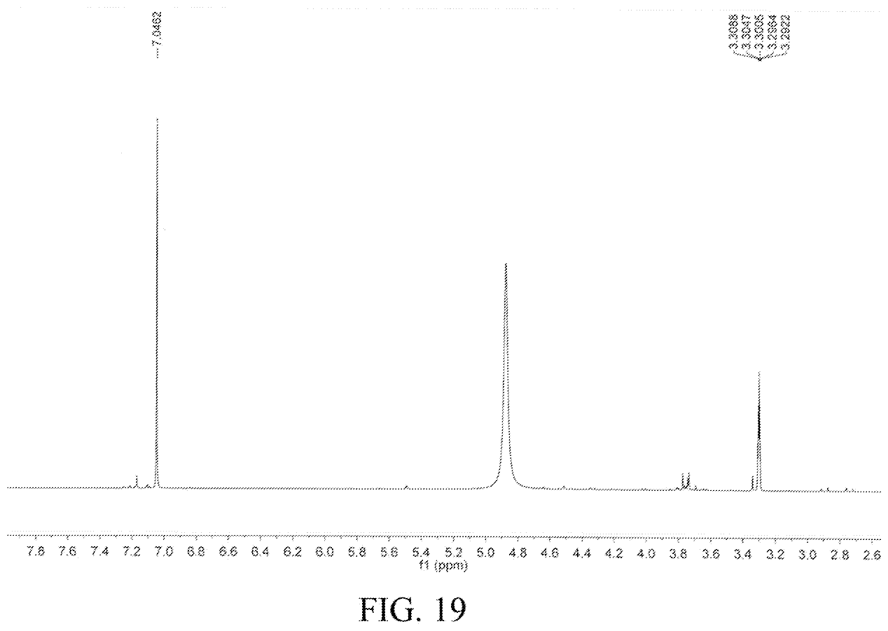

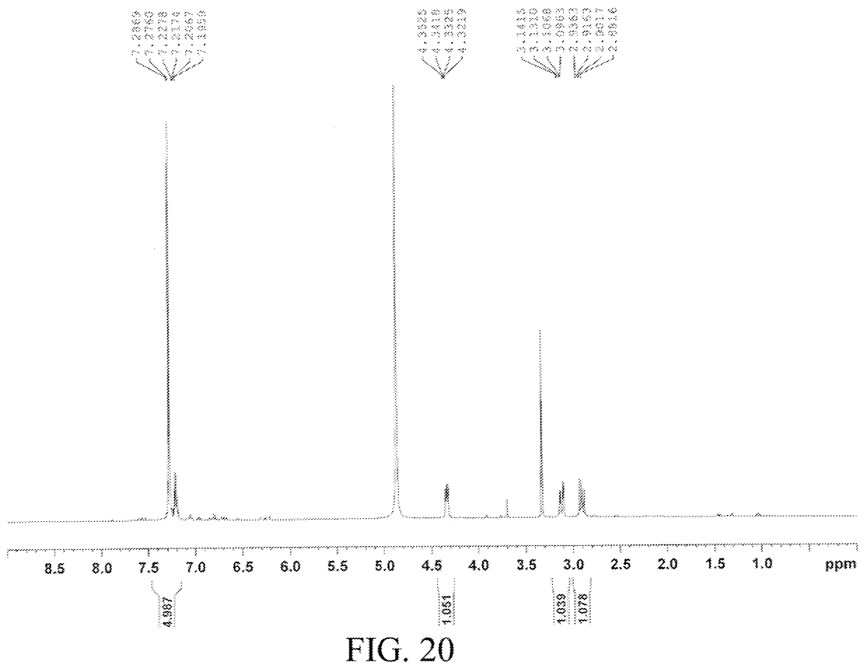

The WF1-2 was purified by normal-phase silica gel column chromatography (with a solvent of methanol and water in a volume ratio of 1:9) to obtain 3.7 mg of compound TCI-LTF-06. After the chemical structure of TCI-LTF-06 was analyzed through ¹H-NMR, TCI-LTF-06 was determined as gallic acid with a molecular mass of 170.12. Its ¹H-NMR spectrum is shown in FIG. 19.

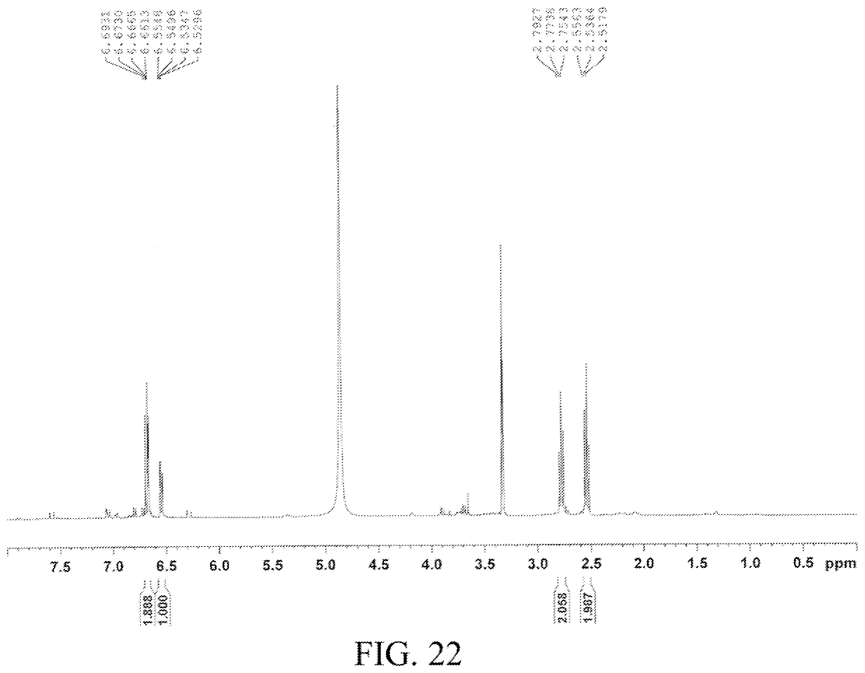

The WF2-4 was purified by normal-phase silica gel column chromatography (with a solvent of methanol and water in a volume ratio of 3:17) to obtain 1.7 mg of compound TCI-LTF-09. After the chemical structure of TCI-LTF-09 was analyzed through ¹H-NMR, TCI-LTF-09 was determined as dihydrocaffeic acid with a molecular mass of 182.176. Its ¹H-NMR spectrum is shown in FIG. 22.

The WF2-2 was purified by normal-phase silica gel column chromatography (with a solvent of methanol and water in a volume ratio of 1:4) to obtain 1.6 mg of compound TCI-LTF-01. After the chemical structure of TCI-LTF-01 was analyzed through ¹H-NMR, TCI-LTF-01 was determined as syringin with a molecular mass of 372.372. Its ¹H-NMR spectrum is shown in FIG. 14.

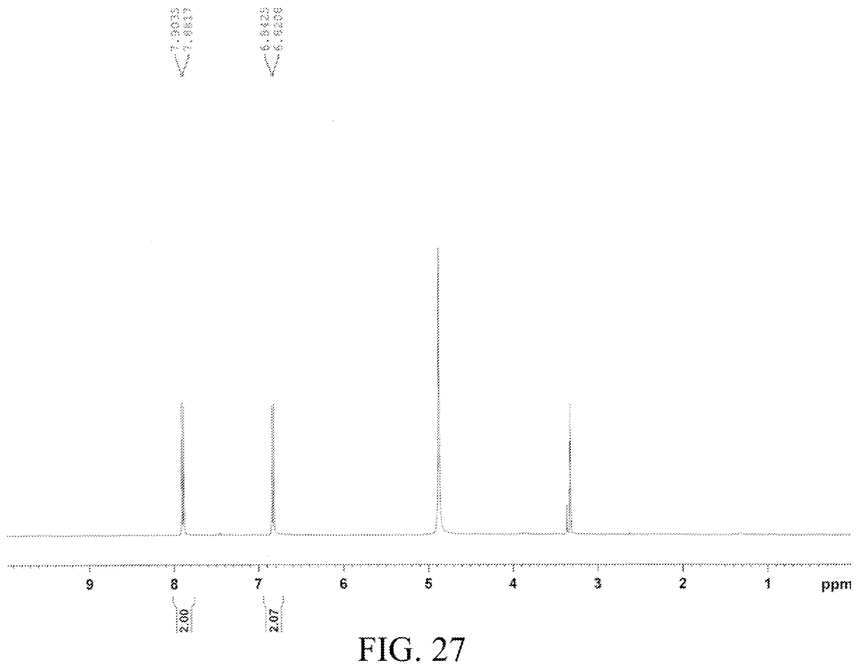

The WF2-3 was purified by normal-phase silica gel column chromatography (with a solvent of methanol and water in a volume ratio of 1:3) to obtain 2 mg of compound TCI-LTF-14. After the chemical structure of TCI-LTF-14 was analyzed through ¹H-NMR, TCI-LTF-14 was determined as 4-hydroxyphenolic acid with a molecular mass of 138.121. Its ¹H-NMR spectrum is shown in FIG. 27.

The WF2-4 was purified by normal-phase silica gel column chromatography (with a solvent of methanol and water in a volume ratio of 3:7) to obtain 6.8 mg of compound TCI-LTF-07. After the chemical structure of TCI-LTF-07 was analyzed through ¹H-NMR, TCI-LTF-07 was determined as 3-phenyllactic acid with a molecular mass of 168. Its ¹H-NMR spectrum is shown in FIG. 20.

In addition, 20 g of BuF was subjected to Sephadex LH-20 column chromatography to obtain a plurality of BuF eluted substances. The eluant used was methanol. Then, the plurality of BuF eluted substances were chromatographed by thin-layer chromatography (TLC aluminium sheets, Silica gel 60 F254, 0.25 mm, Merck, Germany), and the BuF eluted substances with similar results were combined, to obtain 6 fractions (BuF1 to BuF6).

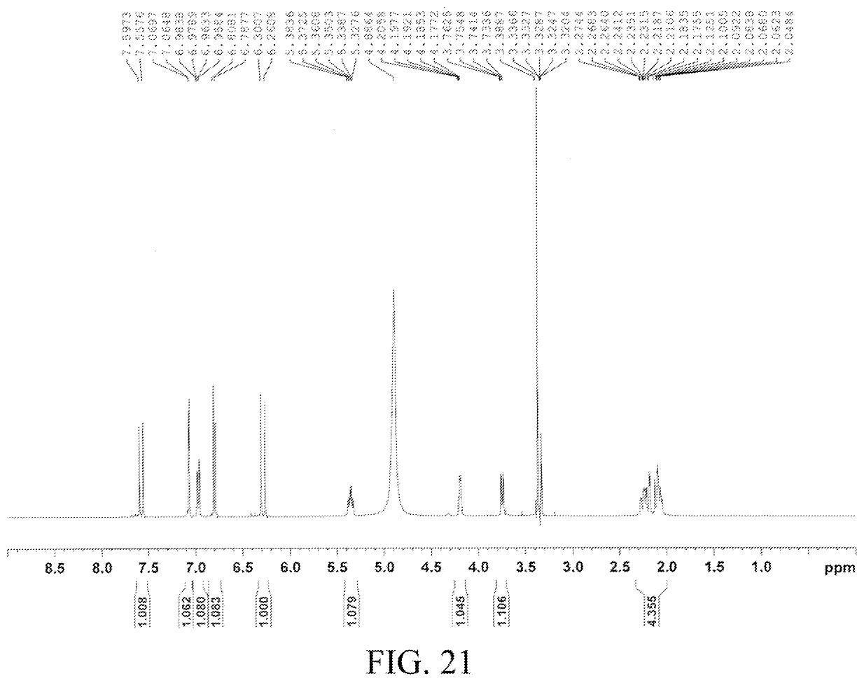

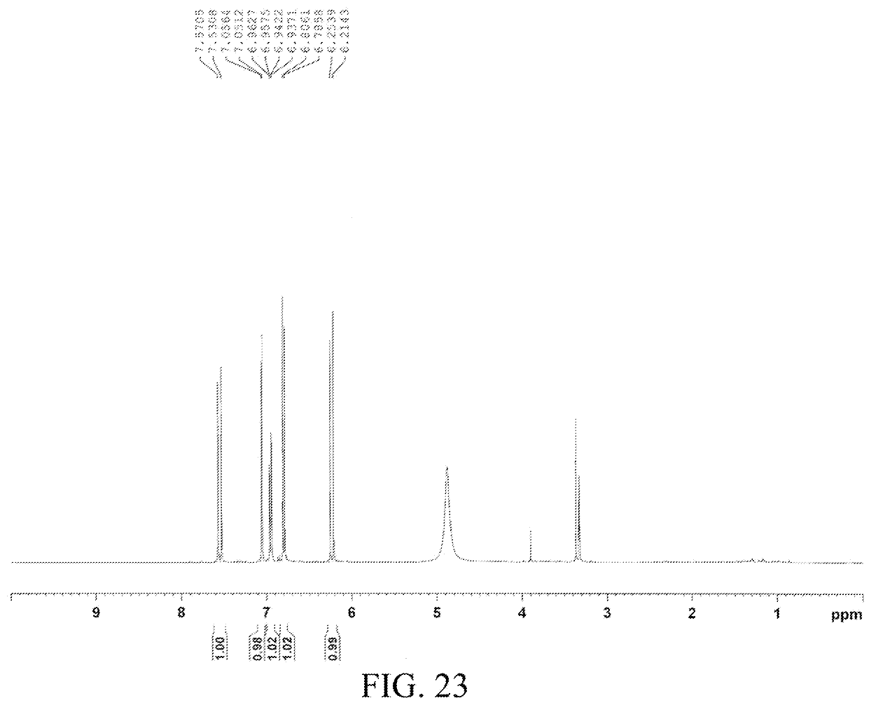

The BuF1 was subjected to reverse-phase HPLC purification (with a solvent of methanol and water in a volume ratio of 1:4) to obtain 8.3 mg of compound TCI-LTF-08 and 4.8 mg of compound TCI-LTF-10. After the chemical structures of TCI-LTF-08 and TI-LTF-10 were analyzed through ¹H-NMR, TI-LIT-08 was determined as chlorogenic acid with a molecular mass of 354.31, and its ¹H-NMR spectrum is shown in FIG. 21; and TCI-LTF-10 was determined as caffeic acid with a molecular mass of 180.16, and its ¹H-NMR spectrum is shown in FIG. 23.

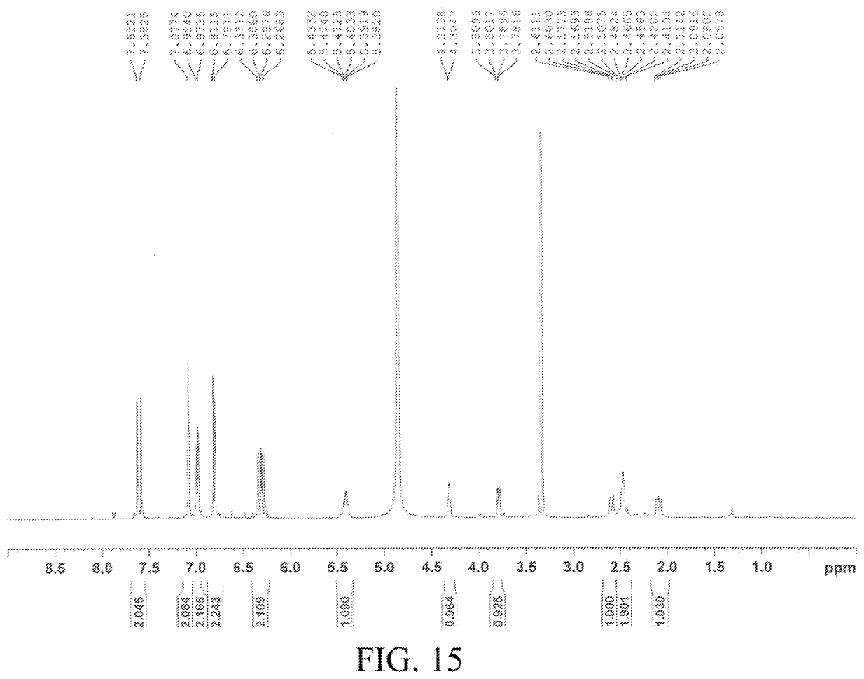

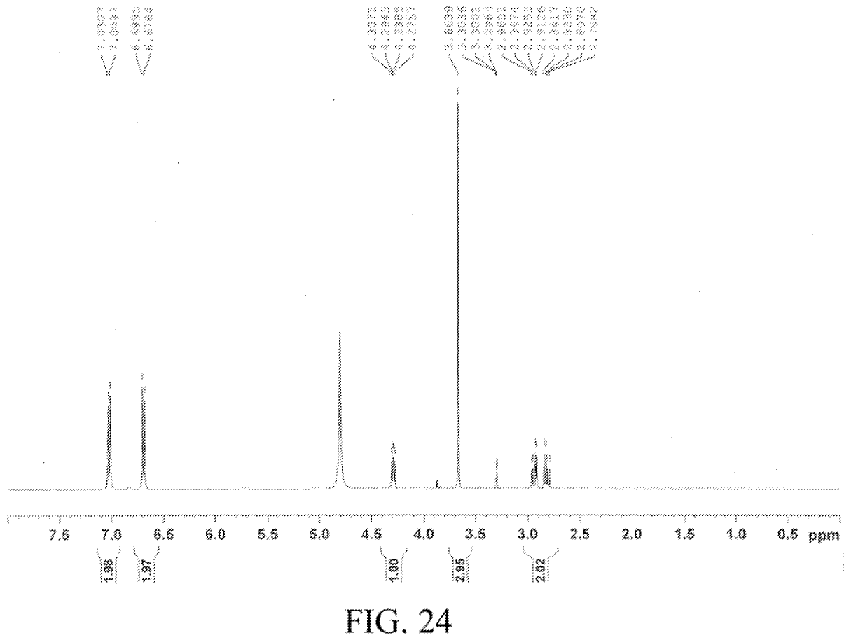

The BuF2 was subjected to reverse-phase HPLC purification (with a solvent of methanol and water in a volume ratio of 1:3) to obtain 1.6 mg of compound TCI-LTF-02 and 3.1 mg of compound TCI-LTF-11. After the chemical structures of TCI-LIT-02 and TCI-LTF-11 were analyzed through ¹H-NMR, TCI-LTF-02 was determined as 1,5-di-o-caffroylquinic acid with a molecular mass of 516.458, and its ¹H-NMR spectrum is shown in FIG. 15; and TCI-LTF-11 was determined as 4-hyderoxy-3-phenlylactic acid methyl ester with a molecular mass of 182, and its ¹H-NMR spectrum is shown in FIG. 24.

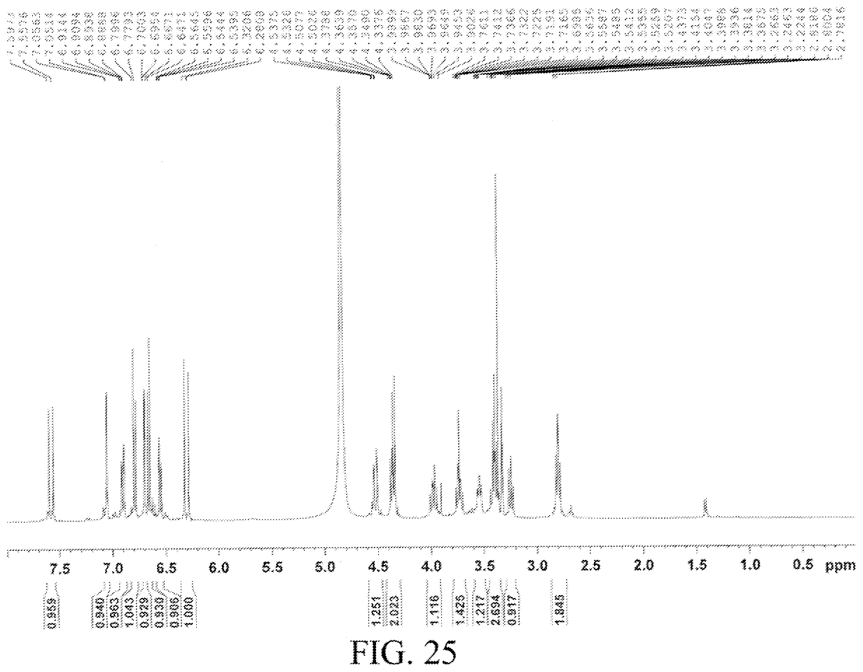

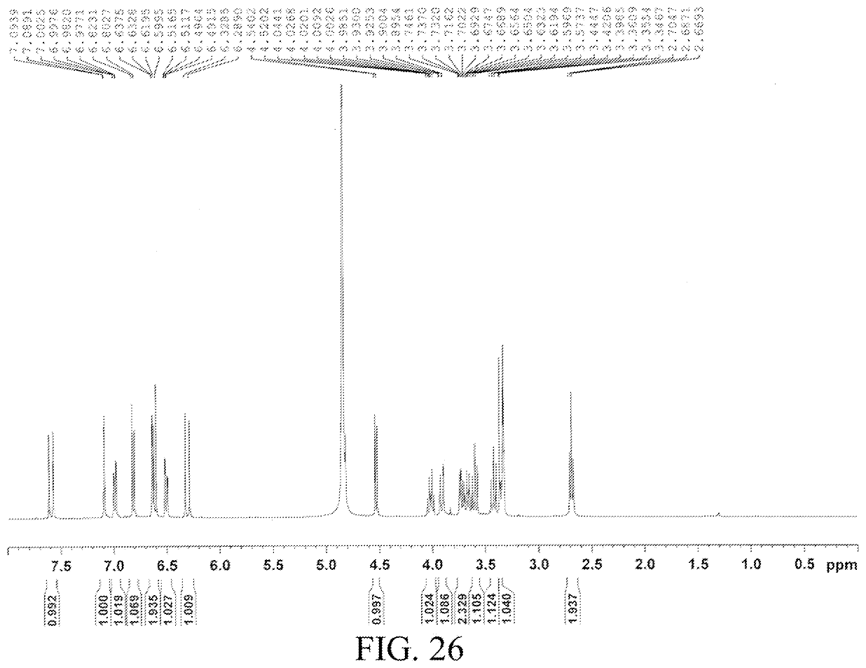

The BuF3 was subjected to reverse-phase HPLC purification (with a solvent of methanol and water in a volume ratio of 3:7) to obtain 1.6 mg of compound TCI-LTF-12 and 1.5 mg of compound TCI-LTF-13. After the chemical structures of TCI-LTF-12 and TCI-LTF-13 were analyzed through ¹H-NMR, TCI-LTF-12 was determined as calceolarioside B with a molecular mass of 478.453, and its ¹H-NMR spectrum is shown in FIG. 25; and TCI-LTF-13 was determined as plantainoside B with a molecular mass of 478.453, and its ¹H-NMR spectrum is shown in FIG. 26.

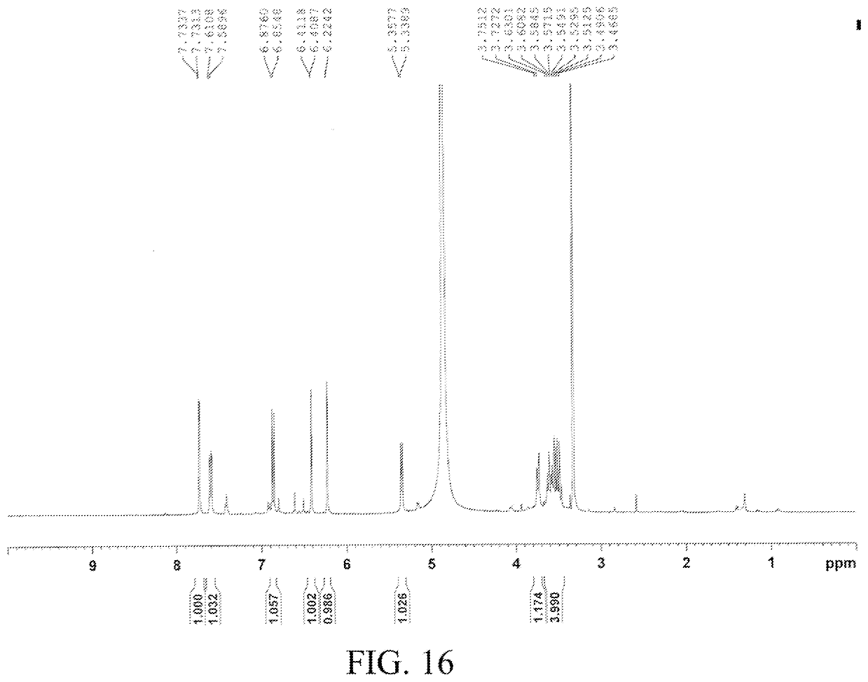

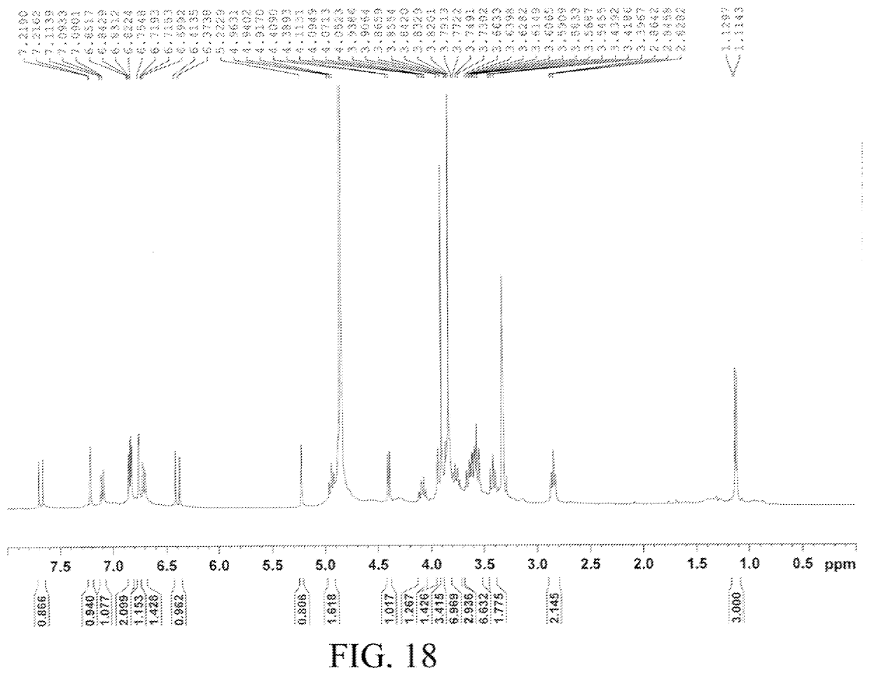

The BuF4 was subjected to reverse-phase HPLC purification (with a solvent of methanol and water in a volume ratio of 2:3) to obtain 1.6 mg of compound TCI-LTF-03 and 1.7 mg of compound TCI-LTF-05. After the chemical structures of TCI-LTF-03 and TCI-LTF-05 were analyzed through ¹H-NMR, TCI-LTF-03 was determined as quercetin-3-glucuronide with a molecular mass of 478.366, and its ¹H-NMR spectrum is shown in FIG. 16; and TCI-LTF-05 was determined as cistanoside D with a molecular mass of 652.85, and its ¹H-NMR spectrum is shown in FIG. 18.

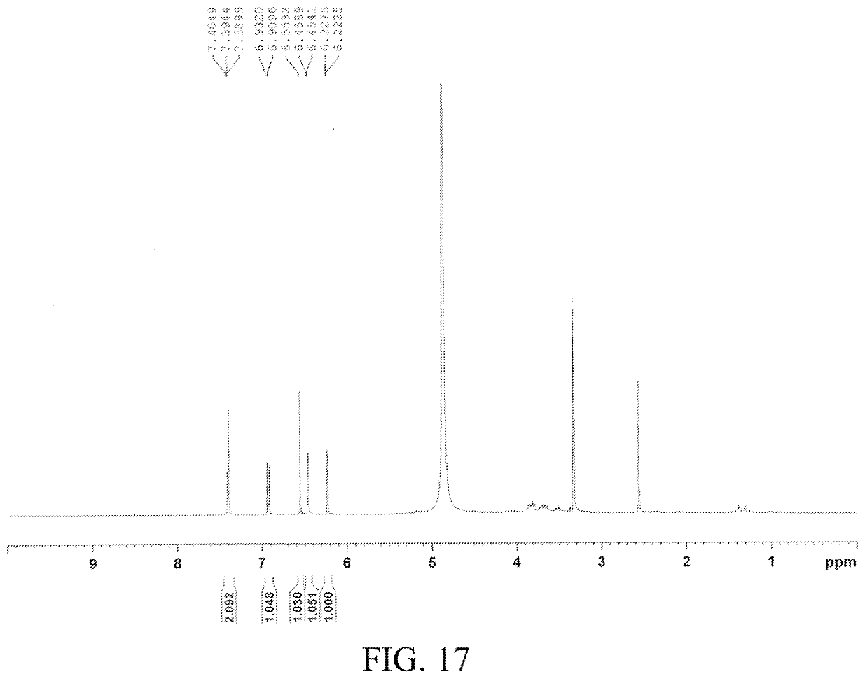

The BuF52 was subjected to reverse-phase HPLC purification (with a solvent of methanol and water in a volume ratio of 1:3) to obtain 1.4 mg of compound TCI-LTF-04. After the chemical structure of TCI-LTF-04 was analyzed through ¹H-NMR, TCI-LTF-04 was determined as luteolin with a molecular mass of 286.241. Its ¹H-NMR spectrum is shown in FIG. 17.

It can be learned from the analysis results in Example 6 and Example 7 that the biologically active substances of the plant ferment are different from those of the unfermented plant aqueous extract, and the plant ferment contains various biologically active substances such as mannitol, gallic acid, dihydrocaffeic acid, syringin, 4-hydroxyphenolic acid, 3-phenyllactic acid, chlorogenic acid, caffeic acid, 1,5-di-o-caffroylquinic acid, 4-hyderoxy-3-phenlylactic acid methyl ester, calceolarioside B, plantainoside B, quercetin-3-glucuronide, cistanoside D, and luteolin, as shown in Table 3.

TABLE 3
Biologically active substance-Chemical name and
chemical structure of compound
TCI-LTF-01
Syringin

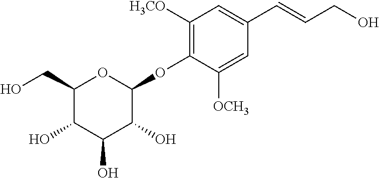 [SMILES: COc1cc(/C=C/CO)cc(OC)c1O[C@@H]1O[C@H](CO)[C@@H](O)[C@H](O)[C@H]1O]

TCI-LTF-02
1,5-Di-O-caffroylquinic acid

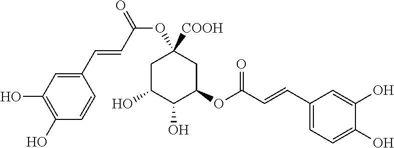 [SMILES: O=C(/C=C/c1ccc(O)c(O)c1)O[C@@H]1C[C@](OC(=O)/C=C/c2ccc(O)c(O)c2)(C(=O)O)C[C@@H](O)[C@H]1O]

TCI-LTF-03
Quercetin-3-glucuronide

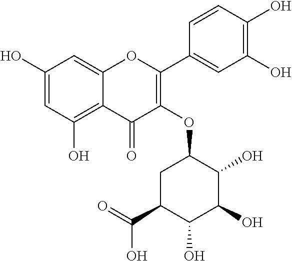 [SMILES: O=C(O)[C@H]1C[C@@H](Oc2c(-c3ccc(O)c(O)c3)oc3cc(O)cc(O)c3c2=O)[C@H](O)[C@@H](O)[C@@H]1O]

TCI-LTF-04
Luteolin

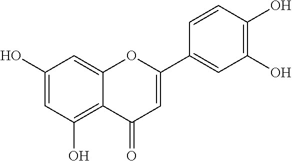 [SMILES: O=c1cc(-c2ccc(O)c(O)c2)oc2cc(O)cc(O)c12]

TCI-LTF-05
Cistanoside D

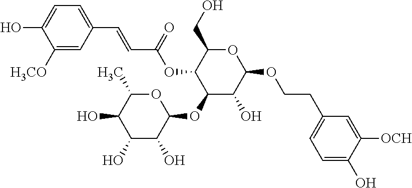

TCI-LTF-06
Gallic acid

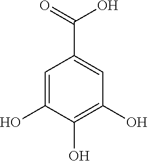 [SMILES: O=C(O)c1cc(O)c(O)c(O)c1]

TCI-LTF-07
3-Phenyllactic acid

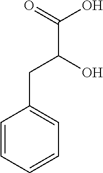 [SMILES: O=C(O)C(O)Cc1ccccc1]

TCI-LTF-08
Chlorogenic acid

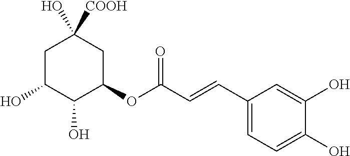 [SMILES: O=C(/C=C/c1ccc(O)c(O)c1)O[C@@H]1C[C@](O)(C(=O)O)C[C@@H](O)[C@H]1O]

TCI-LTF-09
Dihydrocaffeic acid

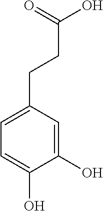 [SMILES: O=C(O)CCc1ccc(O)c(O)c1]

TCI-LTF-10
Caffeic acid

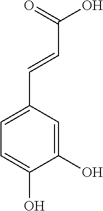 [SMILES: O=C(O)/C=C/c1ccc(O)c(O)c1]

TCI-LTF-11
4-Hyderoxy-3-phenlylactic acid methyl ester

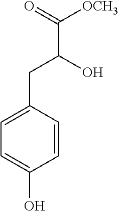 [SMILES: COC(=O)C(O)Cc1ccc(O)cc1]

TCI-LTF-12
Calceolarioside B

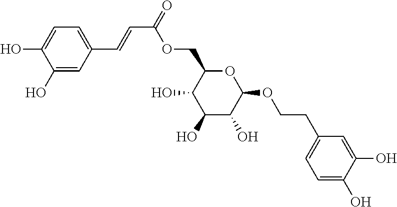 [SMILES: O=C(/C=C/c1ccc(O)c(O)c1)OC[C@H]1O[C@@H](OCCc2ccc(O)c(O)c2)[C@H](O)[C@@H](O)[C@@H]1O]

TCI-LTF-13
Plantainoside B

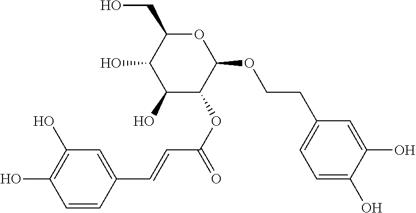 [SMILES: O=C(/C=C/c1ccc(O)c(O)c1)O[C@H]1[C@H](OCCc2ccc(O)c(O)c2)O[C@H](CO)[C@@H](O)[C@@H]1O]

TCI-LTF-14
4-Hydroxyphenolic acid

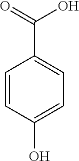

TCI-LTF-15
Mannitol

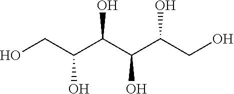 [SMILES: OC[C@@H](O)[C@@H](O)[C@H](O)[C@H](O)CO]

In addition, the ¹H-NMR spectra of the 15 compounds from TCI-LTF-01 to TCI-LTF-15 shown in Table 3 are shown in FIG. 14 to FIG. 28. Moreover, the ¹³C-NMR spectrum of the compound TCI-LTF-15 is shown in FIG. 29.

### Example 8: Analysis on the Effect of Biologically Active Substances in Plant Ferment

### Example 8-1: Experimental Analysis of the Appetite Suppressant Effect

Herein, the expression level of the gene (Gene ID: 5697) of the peptide tyrosine-tyrosine (PPY) protein in human colorectal adenocarcinoma NCI-H716 cells (purchased from the Bioresource Collection and Research Center (BCRC), number 60517; hereinafter referred to as NCI-H716 cells) treated with each of the compounds TCI-LTF-01, TCI-LTF-02, TCI-LTF-04, TCI-LTF-08, TCI-LTF-09, TCI-LTF-10, TCI-LTF-11, TCI-LTF-12, and TCI-LTF-14 that were identified in Example 7 was determined by using the RNA extraction kit, SuperScript® III Reverse Transcriptase, KAPA SYBR®FAST qPCR reagent kit, and quantitative PCR machine. Herein, the PYY protein acts through the NPY receptor, and is used to inhibit human gastric motility and increase colonic absorption of water and electrolytes; and the PYY protein can also be used to inhibit pancreatic secretion, and it has been shown to reduce appetite.

A cell culture medium used is a solution containing 20% of fetal bovine serum (FBS, purchased from Gibco, number 10438-026, US), 1% of antibiotic-antimycotic (purchased from Gibco, number 15240-062), 10 mM of HEPES buffer (purchased from Gibco, number 15630080), and 1 mM of sodium pyruvate (purchased from Gibco, number 11360070). The compounds TCI-LTF-01, TCI-LTF-02, TCI-LTF-04, TCI-LTF-08, TCI-LTF-09, TCI-LTF-10, TCI-LTF-11, TCI-LTF-12, and TCI-LTF-14 each was formulated into 100 mM of stock sample with the DMSO solvent.

First, the NCI-H716 cells were inoculated into a 6-well culture plate containing 2 mL of culture medium in a density of 1×10⁵ cells per well, and cultured at 37° C. for 24 h. Next, the NCI-H716 cells were divided into a blank group and 9 experimental groups. Then, the cell culture medium in each group was replaced with 2 mL of experimental culture medium per well, and then cultured at 37° C. for 12 h. The experimental culture medium in the blank group was a cell culture medium with no treatment (that is, no additional compounds were added into the cell culture medium). The experimental culture medium in each of the 9 experimental groups was a cell culture medium containing 100 mM of corresponding compound identified in Example 7, and the 9 compounds were: syringin (TCI-LTF-01), 1,5-di-o-caffroylquinic acid (TCI-LTF-02), luteolin (TCI-LTF-04), chlorogenic acid (TCI-LTF-08), dihydrocaffeic acid (TCI-LTF-09), caffeic acid (TCI-LTF-10), 4-hyderoxy-3-phenlylactic acid methyl ester (TCI-LTF-11), calceolarioside B (TCI-LTF-12), and 4-hydroxyphenolic acid (TCI-LTF-14). Herein, each of the 9 compounds was first formulated into 100 mM of stock sample with the DMSO solvent, and then added into the cell culture medium to give a final concentration of 100 μM in the cell culture medium. Each group was repeated for three times.

The cell membranes of the NCI-H716 cells treated in each group were broken with a cell lysis buffer to form a cell solution. Next, RNA of the 10 groups of cell solutions was extracted separately by using an RNA extraction reagent kit (purchased from Geneaid, Taiwan, Lot No. FC24015-G). Then, 1000 ng of the extracted RNA in each group was used as a template, and the extracted RNA was reverse transcribed into corresponding cDNA by the SuperScript® III reverse transcriptase (purchased from Invitrogene, USA, number 18080-051). Subsequently, the quantitative real-time reverse transcription polymerase chain reaction was carried out on the 10 groups of cDNA with the primers (SEQ ID NO: 15 to SEQ ID NO: 16) in Table 4 by using the ABI StepOnePlus™ Real-Time PCR system (Thermo Fisher Scientific, USA) and the KAPA SYBR FAST (purchased from Sigma, USA, number 38220000000) to observe the expression level of the PPY gene in the NCI-H716 cells in the 10 groups. The instrument setting conditions for the quantitative real-time reverse transcription polymerase chain reaction were 95° C. for 20 s, 95° C. for 3 s, 60° C. for 30 s, a total of 40 cycles, and gene quantification was carried out by the 2^{-ΔCt} method (using the m-ACTB gene as an internal reference gene). Herein, the quantitative real-time reverse transcription polymerase chain reaction with cDNA can indirectly quantify the expression level of the PYY protein encoded by the PPY gene.

TABLE 4

| Primer name | Sequence number | Primer sequence |
|---|---|---|
| PPY-F | SEQ ID NO: 15 | ATTTGCATACGCACTCCCGA |
| PPY-R | SEQ ID NO: 16 | TTTTGGGACCAGGGAAGGAC |

R represents REVERSE, and F represents FORWARD.

It is to be noted that the gene expression of the PPY gene shown in the figures below is presented in relative magnification. The standard deviation is calculated by using the STDEV formula of Excel software, and whether there is a statistically significant difference is analyzed by one-tailed student t-test in Excel software. In the figures, “*” represents a p value less than 0.05, “**” represents a p value less than 0.01, and “***” represents a p value less than 0.001. More “*” represents more significant statistical differences.

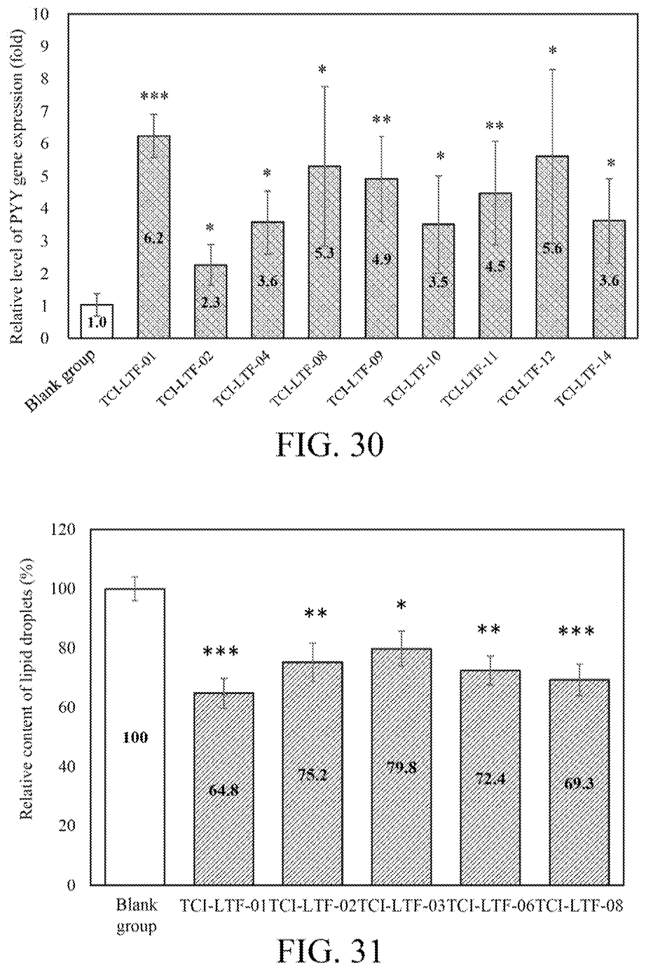

Referring to FIG. 30, the 9 experimental groups were named TCI-LTF-01, TCI-LTF-02, TCI-LTF-04, TCI-LTF-08, TCI-LTF-09, TCI-LTF-10, TCI-LTF-11, TCI-LTF-12, and TCI-LTF-14 respectively. When the expression level of the PYY gene in the blank group is regarded as 1.0, the expression level of the PYY gene in the TCI-LTF-01 experimental group is 6.2, the expression level of the PYY gene in the TCI-LTF-02 experimental group is 2.3, the expression level of the PYY gene in the TCI-LTF-04 experimental group is 3.6, the expression level of the PYY gene in the TCI-LTF-08 experimental group is 5.3, the expression level of the PYY gene in the TCI-LTF-09 experimental group is 4.9, the expression level of the PYY gene in the TCI-LTF-10 experimental group is 3.5, the expression level of the PYY gene in the TCI-LTF-11 experimental group is 4.5, the expression level of the PYY gene in the TCI-LTF-12 experimental group is 5.6, and the expression level of the PYY gene in the TCI-LTF-14 experimental group is 3.6. It can be learned that, compared with the blank group, the PYY gene in the 9 experimental groups is significantly increased. In other words, the plant ferment contains at least 9 compounds that can increase the expression level of the PYY gene in cells of a subject, thereby helping the subject to suppress appetite, indicating that the plant ferment has the effect of suppressing appetite.

### Example 8-2: Analysis on Experiment of Inhibiting Lipid Droplet Accumulation

Herein, the content of glycerol in fat cells is used as a quantitative indicator to observe whether there is lipolysis.

A cell culture medium used is the α-minimum essential medium (α-MEM, Gibco, number 12000-022) containing 20% of fetal bovine serum (FBS. Gibco, number 10438-026) and 1% of antibiotic-antimycotic (Gibco, number 15240-062).

First, 8×10⁴ OP9 cells (purchased from the American Type Culture Collection, ATCC®, number ATCC CRL-2749) were inoculated into a 24-well culture plate containing 500 μL of cell culture medium per well, and cultured at 37° C. for 7 days. During the 7-day cell culture, the cell culture medium was changed every 3 days. After the 7 days of culture, the formation of lipid droplets in the OP9 cells was observed by using a microscope (ZEISS; magnification 400×) to confirm that the OP9 cells were fully differentiated into fat cells for subsequent experiments.

Then, the differentiated fat cells were divided into a blank group and 5 experimental groups. The cell culture medium in each group was replaced with 500 μL of experimental medium per well, and then cultured at 37° C. for 7 days. During the 7-day culture, the experimental medium was replaced with a fresh 500 μL one every 3 days. The experimental culture medium in the blank group was just a cell culture medium (without other compounds), while the experimental culture medium in each of the 5 experimental group was a cell culture medium containing 100 mM of corresponding compound identified in Example 7, and the 5 compounds were: syringin (TCI-LTF-01), 1,5-di-o-caffroylquinic acid (TCI-LTF-02), quercetin-3-glucuronide (TCI-LTF-03), gallic acid (TCI-LTF-06), and chlorogenic acid (TCI-LTF-08). Herein, each of the 5 compounds was first formulated into 100 mM of stock sample with the DMSO solvent, and then added into the cell culture medium to give a final concentration of 100 μM in the cell culture medium.

The content of glycerol was determined by using a glycerol cell-based assay kit (purchased from Cayman, USA, product number 10011725) according to the following steps. The experimental culture medium (that is, the experimental culture medium in which fat cells had been cultured, without the fat cells) in each group was collected, 25 μL of the experimental culture medium was transferred into a new 96-well culture plate, 100 μL of reconstituted free glycerol assay reagent was added into each well to react at room temperature for 15 min, and the absorbance OD_{540 nm} of each group was read from the 96-well culture plate by an ELISA reader, to quantify the content of glycerol released into the experimental culture medium through lipolysis, as shown in FIG. 31. Herein, the content of glycerol is directly proportional to the amount of lipolysis. The obtained results were analyzed by student t-test using Excel software to determine whether there is a statistically significant difference between two sample groups. (In the figures, “*” represents a p value less than 0.05, “**” represents a p value less than 0.01, and “***” represents a p value less than 0.001. More “*” represents more significant statistical differences.)

Refer to FIG. 31. Herein, the 5 experimental groups were named after the added compounds: TCI-LTF-01, TCI-LTF-02, TCI-LTF-03, TCI-LTF-06, and TCI-LTF-08. When the lipid droplet content of the blank group is regarded as 100%, the lipid droplet content of the TCI-LTF-01 experimental group is 64.8% (that is, reduced by 35.2%), the lipid droplet content of the TCI-LTF-02 experimental group is 75.2% (that is, reduced by 24.8%), the lipid droplet content of the TCI-LTF-03 experimental group is 79.8% (that is, reduced by 20.2%), the lipid droplet content of the TCI-LTF-06 experimental group is 72.4% (that is, reduced by 27.6%), and the lipid droplet content of the TCI-LTF-08 experimental group is 69.3% (that is, reduced by 30.7%). In other words, compared with the blank group, the lipid droplet content in fat cells of the 5 experimental groups is significantly reduced. It can be learned that the plant ferment contains at least 5 compounds that can effectively promote lipolysis, indicating that the plant ferment has the function of improving fat metabolism of subjects, thereby achieving fat loss.

Based on the above, the plant ferment extracted from the mulberry, pomegranate, purslane, wild bitter melon, and fennel and fermented with the yeast, Lactobacillus plantarum, and Acetobacter aceti according to any embodiment of the present invention can be used to prepare a composition for weight loss. In some embodiments, the composition is effectively involved in regulating the process of eating and fat storage of a subject, and can reduce the appetite of the subject, increase the time between meals of the subject, reduce hunger of the subject, and/or reduce the number of meals other than dinner of the subject per week. In some embodiments, the composition contributes to the effective reduction of fat stored in cells by increasing an expression level of fat metabolism genes in cells and/or reducing an expression level of fat accumulation genes, thereby achieving the effects of reducing body weight, BMI, whole body fat, and/or hip circumference. In some embodiments, the composition may also reduce insulin resistance, and increase the sensitivity of body cells to glucose, so that excess energy is less likely to be stored as fat, to reduce fat accumulation. Based on this, the plant ferment of any embodiment can lose fat, reduce the appetite, increase the time between meals, reduce hunger, reduce the number of meals other than dinner per week, reduce insulin resistance, and reduce fat stored in cells, thereby achieving the effects of reducing body weight, BMI, whole body fat, and/or hip circumference.

Although the present invention has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. A plant ferment, prepared by:
mixing a mulberry (Morus alba), a pomegranate (Punica granatum), a purslane (Portulaca oleracea), a wild bitter melon (Momordica charantia var. abbreviata), and a fennel (Foeniculum vulgare) in a ratio of (0.5-4):(4-8):(0.5-4):(0.1-2):(0.5-4) to form a mixture;
extracting the mixture with a solvent to obtain a plant extract; and
fermenting the plant extract to obtain the plant ferment.

2. The plant ferment according to claim 1, wherein the plant extract is obtained by extracting the mixture comprising the mulberry, the pomegranate, the purslane, the wild bitter melon, and the fennel with water as the solvent at 5-100° C. for 0.5-2 h.

3. The plant ferment according to claim 2, wherein the mixture and water are mixed in a ratio of (10-15):(80-90).

4. The plant ferment according to claim 1, obtained by fermenting the plant extract with yeast (Saccharomyces cerevisiae), Lactobacillus plantarum, and Acetobacter aceti.

5. The plant ferment according to claim 4, wherein the yeast is added in an amount of 0.01-0.5% (w/w); the Lactobacillus plantarum is added in an amount of 0.01-0.2% (w/w); and the Acetobacter aceti is added in an amount of 1-10% (w/w).

6. A weight loss method, comprising: administering to a subject in need thereof an effective dose of a composition, wherein the composition comprises a plant ferment prepared by: mixing a mulberry (Morus alba), a pomegranate (Punica granatum), a purslane (Portulaca oleracea), a wild bitter melon (Momordica charantia var. abbreviata), and a fennel (Foeniculum vulgare) in a ratio of (0.5-4):(4-8):(0.5-4):(0.1-2):(0.5-4) to form a mixture; extracting the mixture with a solvent to obtain a plant extract; and fermenting the plant extract to obtain the plant ferment.

7. The method according to claim 6, wherein the composition is capable of reducing the appetite, increasing the time between meals, reducing hunger, and/or reducing the number of meals other than dinner per week.

8. The method according to claim 6, wherein the composition is capable of reducing body weight, body mass index (BMI), whole body fat, and/or hip circumference.

9. The method according to claim 6, wherein the composition is capable of reducing insulin resistance.

10. The method according to claim 6, wherein the plant ferment increases an expression level of fat metabolism genes in cells and/or reduces an expression level of fat accumulation genes in cells.

11. The method according to claim 10, wherein the fat metabolism gene is selected from ATGL, LIPE, UCP1, UCP2, and a combination thereof.

12. The method according to claim 10, wherein the fat accumulation gene is PLIN1 and/or PPARG2.

13. The method according to claim 6, wherein the plant ferment is further prepared into a food composition, a dietary supplement composition, or a skin topical agent.


SEQUENCE LISTING (16 sequences)

SEQ ID NO: 1 - DNA, 20 bp, Artificial Sequence
  ATGL-F
ggatggcggcatttcagaca

SEQ ID NO: 2 - DNA, 21 bp, Artificial Sequence
  ATGL-R
caaagggttgggttggttcag

SEQ ID NO: 3 - DNA, 20 bp, Artificial Sequence
  LIPE(HSL)-F
tggcacaccattttgacctg

SEQ ID NO: 4 - DNA, 21 bp, Artificial Sequence
  LIPE(HSL)-R
ttgcggttagaagccacatag

SEQ ID NO: 5 - DNA, 21 bp, Artificial Sequence
  UCP1-F
aggcttccagtaccattaggt

SEQ ID NO: 6 - DNA, 22 bp, Artificial Sequence
  UCP1-R
ctgagtgaggcaaagctgattt

SEQ ID NO: 7 - DNA, 21 bp, Artificial Sequence
  UCP2-F
atggttggtttcaaggccaca

SEQ ID NO: 8 - DNA, 22 bp, Artificial Sequence
  UCP2-R
cggtatccagagggaaagtgat

SEQ ID NO: 9 - DNA, 19 bp, Artificial Sequence
  PLIN1-F
gggacctgtgagtgcttcc

SEQ ID NO: 10 - DNA, 23 bp, Artificial Sequence
  PLIN1-R
gtattgaagagccgggatctttt

SEQ ID NO: 11 - DNA, 20 bp, Artificial Sequence
  PPARG2-F
tcgctgatgcactgcctatg

SEQ ID NO: 12 - DNA, 21 bp, Artificial Sequence
  PPARG2-R
gagaggtccacagagctgatt

SEQ ID NO: 13 - DNA, 20 bp, Artificial Sequence
  m-ACTB-F
ggctgtattcccctccatcg

SEQ ID NO: 14 - DNA, 22 bp, Artificial Sequence
  m-ACTB-R
ccagttggtaacaatgccatgt

SEQ ID NO: 15 - DNA, 20 bp, Artificial Sequence
  PPY-F
atttgcatacgcactcccga

SEQ ID NO: 16 - DNA, 20 bp, Artificial Sequence
  PPY-R
ttttgggaccagggaaggac